(19) United States
(12) Patent Application Publication
(10) Pub. No.: US 2023/0227790 A1
(43) Pub. Date: Jul. 20, 2023

(54) METHOD OF ASSEMBLYING TWO-COMPONENT VIRUS-LIKE PARTICLE

(71) Applicants: Scot R. SHEPARD, Chapel Hill, NC (US); Hans R. LIEN, Bozeman, MT (US); Ross M. TAYLOR, Seattle, WA (US); Icosavax, Inc., Seattle, WA (US)

(72) Inventors: Scot R. SHEPARD, Seattle, WA (US); Hans R. LIEN, Bozeman, MT (US); Ross M. TAYLOR, Seattle, WA (US); Charles RICHARDSON, Seattle, WA (US)

(21) Appl. No.: 18/000,566

(22) PCT Filed: Jun. 9, 2021
(86) PCT No.: PCT/US2021/036689

§ 371 Date: Dec. 2, 2022

Related U.S. Application Data
(60) Provisional application No. 63/036,505, filed on Jun. 9, 2020.

(51) Int. Cl. C12N 7/00; A61K 39/12; C07K 14/16

(52) U.S. Cl. CPC C12N 7/00; A61K 39/12; C07K 14/16; A61K 2039/5258

(57) ABSTRACT

Disclosed are methods of a method of making a nanostructure, comprising adding a component A (compA) protein to a solution comprising a component B (compB) protein under conditions that minimize shear stress, thereby forming a compA:compB complex. Further disclosed are methods of making a nanostructure, comprising (i) providing a first inlet fluid stream comprising a first protein and a second inlet fluid stream comprising a second protein, and (ii) contacting the first inlet fluid stream and the second inlet fluid stream to form an outlet stream, wherein mixing of the first protein and the second protein occurs in the outlet stream, thereby forming a protein complex comprises the first protein and the second protein. A microfluidic mixer may be used. The methods may further comprise purifying the compA:compB complex from excess compA, excess compB, and/or other impurities by filtering the solution with a 1,000 kDa membrane or an equivalent thereof.

### CROSS REFERENCE TO RELATED APPLICATION

This application claims priority to U.S. Provisional Application No. 63/036,505, filed Jun. 9, 2020, the entire contents of which are hereby incorporated by reference.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ICVX_009_01WO_ST25.txt. The text file is 838 KB, created on Jun. 9, 2021, and is being submitted electronically via EFS-Web.

### FIELD OF INVENTION

The present disclosure relates generally to self-assembling protein nanostructures, in particular methods of making nanostructures, including nanostructure-based vaccines.

### BACKGROUND

Protein-based Virus-Like Particles (pbVLPs), also called nanostructures, provide a useful platform to present proteins or other macromolecules symmetrically. They can be distinguished from conventional VLPs made from viral capsid proteins (e.g., from a non-enveloped virus) or lipid-embedded proteins (e.g., extracted from an enveloped virus or made using recombinant membrane proteins mixed with lipids). The later do not generally have defined symmetry. The former are generally limited in their ability to display proteins, due to challenges in attaching proteins to viral capsids.

One application for VLPs generally and for pbVLPs in particular is as vaccines. Studies have demonstrated experimentally that antigens displayed on pbVLPs elicit stronger antibody responses than conventional subunit vaccines and than non-symmetric VLPs.

Bale et al., Science 353:389-394 (2016) discloses various two-component icosahedral pbVLPs, including a set of pbVLPs made from protein components designated component A (compA) and component B (compB).

There remains a need in the art for methods of expressing, purifying, and assembling protein-based Virus-Like Particles. The present disclosure fulfills this need.

### SUMMARY OF THE INVENTION

The present invention relates generally to methods to assemble and purify pbVLP.

Provided herein is a method of making a nanostructure, comprising adding a component A (compA) protein to a solution comprising a component B (compB) protein under conditions that minimize shear stress, thereby forming a compA:compB complex. In some embodiments, the compA:compB complex is a complex having icosahedral symmetry. In some embodiments, the compA:compB complex is a pbVLP.

In some embodiments, the disclosure provides a method of making a nanostructure, comprising adding a fusion protein to a solution comprising a component B (compB) protein under conditions that minimize shear stress, wherein the fusion protein comprises a compA protein linked to an antigenic protein, thereby forming a compA:compB complex.

In some embodiments, the conditions that minimize shear stress comprise adding the compA protein to the compB protein under the surface of the solution.

In some embodiments, the conditions that minimize shear stress comprise mixing the solution without mechanical mixing.

In some embodiments, the conditions that minimize shear stress comprise mixing the solution without a stir bar.

In some embodiments, the conditions that minimize shear stress comprise mixing the solution without an impeller.

In some embodiments, the conditions that minimize shear stress comprise mixing the solution using an orbital agitation.

In some embodiments, the conditions that minimize shear stress comprise mixing the solution using a microfluidic mixer. In some embodiments, the microfluidic mixer is a Nanoassembler® Ignite™ cartridge or any equivalent thereof.

In some embodiments, the method comprising adding the compA protein in excess to the compB protein. In some embodiments, the method comprises adding the compA protein to the compB protein, wherein the molar concentration of the compA protein and the molar concentration of the compB protein are substantially equivalent.

In some embodiments, the method provides for mixing of compA and compB without substantial precipitation. In some embodiments, the method provides for mixing of compA and compB without substantial precipitation relative to mechanical mixing of a solution of compA and compB.

In some embodiments, the method provides formation of the compA:compB complex in an amount that is at least about 40% of total protein, e.g., as measured by size exclusion chromatography. In some embodiments, the method provides formation of the compA:compB complex in an amount that about 40%, about 50%, about 60%, about 70%, about 80%, about 85%, about 90%, or about 95% of total protein, e.g., as measured by size exclusion chromatography.

In some embodiments, the method further comprises purifying the compA:compB complex from excess compA, excess compB, and/or other impurities by filtering with the solution with a 1,000 kDa membrane or an equivalent thereof. In some embodiments, the method further comprises purifying the compA:compB complex from excess compA, excess compB, and/or other impurities by filtering with the solution with a hydrophilic membrane having a pore size of about 300 kDa-3,000 kDa. In some embodiments, the pore size is about 800 kDa, about 900 kDa, about 1000 kDa, about 1100 kDa, about 1200 kDa, about 1300 kDa, about 1400 kDa, or about 1500 kDa. In some embodiments, the hydrophilic membrane comprises a material selected from PVD cellulose, composite regenerated cellulose (CRC), polytetrafluoroethylene (PTFE), polyvinylidene fluoride (PVDF), nylon, and polyethersulfone.

In some embodiments, the 1,000 kDa membrane is a composite regenerated cellulose (CRC) membrane.

In some embodiments, the 1,000 kDa membrane is Ultracel® 1000 kDa Membrane or an equivalent thereof.

In some embodiments, the filtering comprises tangential flow filtration (TFF). In some embodiments, the filtering provides the compA:compB complex with purity of about 80% or higher as measured by percent weight of total protein. In some embodiments, the filtering provides the compA:compB complex with purity of about 80%, about 85%, about 90%, about 95%, about 96%, about 97%, about 98%, or about 99% as measured by percent weight of total protein. In some embodiments, the filtering provides the compA:compB complex with purity of about 90-99% as measured by percent weight of total protein.

In some embodiments, the method does not comprise purifying the solution with a hydrophobic membrane (e.g., Pellicon Biomax 1000 kDa), a Butyl Convective Interaction Media (CIM) column, a Captocore700 column, or an equivalent thereto.

In some embodiments, the method is a continuous or semi-continuous process.

In some embodiments, the compA protein is continuously produced prior to the adding step.

In some embodiments, the compB protein is provided as one or more frozen batches.

In some aspects, the disclosure provides a method of making a nanostructure, comprising (i) providing a first inlet fluid stream comprising a first protein and a second inlet fluid stream comprising a second protein, and (ii) contacting the first inlet fluid stream and the second inlet fluid stream to form an outlet stream, wherein mixing of the first protein and the second protein occurs in the outlet stream, thereby forming a protein complex comprising the first protein and the second protein. In some embodiments, the method comprises (i) providing a first inlet fluid stream comprising a first protein and a second inlet fluid stream comprising a second protein, wherein the first protein comprises a compA protein, and wherein the second protein comprises a compB protein; and (ii) contacting the first inlet fluid stream and the second inlet fluid stream to form an outlet stream, wherein mixing of the first protein and the second protein occurs in the outlet stream, thereby forming a protein based VLP (pbVLP) complex comprising the first protein and the second protein.

In some embodiments, the first inlet fluid stream and the second inlet fluid stream are joined in a three-way configuration to form the outlet stream, optionally wherein the three-way configuration is a T-shaped or Y-shaped configuration. In some embodiments, the first inlet fluid stream and the second inlet fluid stream are joined to form the outlet stream, wherein the outlet stream passes through a static mixer (e.g., a pipe mixer), wherein mixing of the first protein (e.g., compA) and the second protein (e.g., compB) occurs as the outlet stream passes through the static mixer, thereby forming a complex (e.g., pbVLP complex) comprising the first protein and the second protein.

In some embodiments, the first inlet fluid stream and the second inlet fluid stream are joined to form the outlet stream using a microfluidic mixer. In some embodiments, the microfluidic mixer is a Nanoassembler® Ignite™ cartridge or any equivalent thereof. In some embodiments, the microfluidic mixer comprises one or more passive mixing elements to facilitate mixing of the first protein and the second protein. In some embodiments, the outlet stream comprises a molar concentration of the first protein (e.g., compA) that exceeds the molar concentration of the second protein (e.g., compB). In some embodiments, the outlet stream comprises a molar concentration of the first protein (e.g., compA) that is substantially equivalent to the molar concentration of the second protein (e.g., compB).

In some embodiments, the mixing of the first protein (e.g., compA) and the second protein (e.g., compB) occurs without substantial precipitation of the first protein, the second protein, the complex, or a combination thereof.

In some embodiments, the complex is formed in an amount that is at least about 40% of total protein, e.g., as measured by size exclusion chromatography. In some embodiments, the method provides formation of the compA:compB complex in an amount that about 40%, about 50%, about 60%, about 70%, about 80%, about 85%, about 90%, or about 95% of total protein, e.g., as measured by size exclusion chromatography.

In some embodiments, the method further comprises purifying the complex from excess second protein, and/or other impurities by filtering the solution with a 1,000 kDa membrane or an equivalent thereof. In some embodiments, the method further comprises purifying the compA:compB complex from excess compA, excess compB, and/or other impurities by filtering with the solution with a hydrophilic membrane having a pore size of about 300 kDa-3,000 kDa. In some embodiments, the pore size is about 800 kDa, about 900 kDa, about 1000 kDa, about 1100 kDa, about 1200 kDa, about 1300 kDa, about 1400 kDa, or about 1500 kDa. In some embodiments, the hydrophilic membrane comprises a material selected from PVD cellulose, composite regenerated cellulose (CRC), polytetrafluoroethylene (PTFE), polyvinylidene fluoride (PVDF), nylon, and polyethersulfone.

In some embodiments, the 1,000 kDa membrane is a composite regenerated cellulose (CRC) membrane.

In some embodiments, the 1,000 kDa membrane is Ultracel® 1000 kDa Membrane or an equivalent thereof.

In some embodiments, the filtering comprises tangential flow filtration (TFF). In some embodiments, the filtering provides the compA:compB complex with purity of about 80% or higher as measured by percent weight of total protein. In some embodiments, the filtering provides the compA:compB complex with purity of about 80%, about 85%, about 90%, about 95%, about 96%, about 97%, about 98%, or about 99% as measured by percent weight of total protein. In some embodiments, the filtering provides the compA:compB complex with purity of about 90-99% as measured by percent weight of total protein.

In some embodiments, the method does not comprise purifying the solution with a hydrophobic membrane (e.g., Pellicon Biomax 1000 kDa), a Butyl Convective Interaction Media (CIM) column, a Captocore700 column, or an equivalent thereto.

In some embodiments, the method is a continuous or semi-continuous process.

In some embodiments, the first protein is continuously produced prior to the adding step.

In some embodiments, the second protein is provided as one or more frozen batches.

In some embodiments, the first component is a component A (compA) described herein and/or the first component is a component B (compB) described herein. In some embodiments, the compA comprises a polypeptide sequence that has at least 90%, at least 95%, at least 99%, or 100% identity to any one of SEQ IN NOs: 1, 3, 5, 7, 9, 11, 13, 15, 17, 19, 21, 23, 25, 26, 29-31, 35, 37, 39, 41, 43, 45, 47, 49, 51, 53, 55, 57, and 59. In some embodiments, the compB comprises a polypeptide sequence that has at least 90%, at least 95%, at least 99%, or 100% identity to any one of SEQ IN NOs: 2, 4, 6, 8, 10, 12, 14, 16, 18, 20, 22, 24, 27, 28, 32-34, 36, 38, 40, 42, 44, 46, 48, 50, 52, 54, 56, and 58. In some embodiments, the compA and the compB each comprise a polypeptide sequence that has at least 90%, at least 95%, at least 99%, or 100% identity to any one of (i) SEQ ID NO:1 and SEQ ID NO:2 respectively; (ii) SEQ ID NO:3 and SEQ ID NO:4 respectively; (iii) SEQ ID NO:3 and SEQ ID NO:24 respectively; (iv) SEQ ID NO:23 and SEQ ID NO:4 respectively; (v) SEQ ID NO:35 and SEQ ID NO:36 respectively; (vi) SEQ ID NO:5 and SEQ ID NO:6 respectively; (vii) SEQ ID NO:5 and SEQ ID NO:27 respectively; (viii) SEQ ID NO:5 and SEQ ID NO:28 respectively; (ix) SEQ ID NO:25 and SEQ ID NO:6 respectively; (x) SEQ ID NO:25 and SEQ ID NO:27 respectively; (xi) SEQ ID NO:25 and SEQ ID NO:28 respectively; (xii) SEQ ID NO:26 and SEQ ID NO:6 respectively; (xiii) SEQ ID NO:26 and SEQ ID NO:27 respectively; (xiv) SEQ ID NO:26 and SEQ ID NO:28 respectively; (xv) SEQ ID NO:37 and SEQ ID NO:38 respectively; (xvi) SEQ ID NO:7 and SEQ ID NO:8 respectively; (xvii) SEQ ID NO:7 and SEQ ID NO:32 respectively; (xviii) SEQ ID NO:7 and SEQ ID NO:33 respectively; (xix) SEQ ID NO:7 and SEQ ID NO:34 respectively; (xx) SEQ ID NO:29 and SEQ ID NO:8 respectively; (xxi) SEQ ID NO:29 and SEQ ID NO:32 respectively; (xxii) SEQ ID NO:29 and SEQ ID NO:33 respectively; (xxiii) SEQ ID NO:29 and SEQ ID NO:34 respectively; (xxiv) SEQ ID NO:30 and SEQ ID NO:8 respectively; (xxv) SEQ ID NO:30 and SEQ ID NO:32 respectively; (xxvi) SEQ ID NO:30 and SEQ ID NO:33 respectively; (xxvii) SEQ ID NO:30 and SEQ ID NO:34 respectively; (xxviii) SEQ ID NO:31 and SEQ ID NO:8 respectively; (xxix) SEQ ID NO:31 and SEQ ID NO:32 respectively; (xxx) SEQ ID NO:31 and SEQ ID NO:33 respectively; (xxxi) SEQ ID NO:31 and SEQ ID NO:34 respectively; (xxxii) SEQ ID NO:39 and SEQ ID NO:40 respectively; (xxxiii) SEQ ID NO:9 and SEQ ID NO:10 respectively; (xxxiv) SEQ ID NO:11 and SEQ ID NO:12 respectively; (xxxv) SEQ ID NO:13 and SEQ ID NO:14 respectively; (xxxvi) SEQ ID NO:15 and SEQ ID NO:16 respectively; (xxxvii) SEQ ID NO:19 and SEQ ID NO:20 respectively; (xxxviii) SEQ ID NO:21 and SEQ ID NO:22 respectively; (xxxviv) SEQ ID NO:23 and SEQ ID NO:24 respectively; (xl) SEQ ID NO:41 and SEQ ID NO:42 respectively; (xli) SEQ ID NO:43 and SEQ ID NO:44 respectively; (xlii) SEQ ID NO:45 and SEQ ID NO:46 respectively; (xliii) SEQ ID NO:47 and SEQ ID NO:48 respectively; (xliv) SEQ ID NO:49 and SEQ ID NO:50 respectively; (xlv) SEQ ID NO:51 and SEQ ID NO:44 respectively; (xlvi) SEQ ID NO:53 and SEQ ID NO:52 respectively; (xlvii) SEQ ID NO:55 and SEQ ID NO:54 respectively; (xlviii) SEQ ID NO:57 and SEQ ID NO:56 respectively; and (xlvix) SEQ ID NO:59 and SEQ ID NO:58 respectively.

In some embodiments, a fusion protein comprises the compA protein, wherein the compA protein is linked to an antigenic protein. In some embodiments, the fusion protein comprises a compA protein linked to an antigenic protein by a linker.

In some embodiments, the antigenic protein is selected from HIV Env, RSV F, EBV gp350, CMV gB, CMV UL128, CMV UL130, CMV UL131A, CMV gH, CMV gL, Lyme OspA, Pertussis toxin, Dengue E, SARS S, MERS S, Zaire ebolavirus GP, Sudan ebolavirus GP, Marburg virus GP, Hanta virus Gn, Hanta virus Gc, HepB surface antigen, Measles H, Zika envelope domain III, Malaria CSP, Malaria Pfs25, Nipah virus F, Nipah virus G, Rotavirus VP4, Rotavirus VP8*, hMPV F, hMPV G, PV F, PV HN, MenB fHbp, MenB NadA, coronavirus S protein, coronavirus RBD, and MenB NHBA. In some embodiments, the antigenic protein comprises a paramyxovirus and/or penumovirus F protein or an antigenic fragment thereof. In some embodiments, the antigenic protein comprises a respiratory syncytial virus (RSV) F protein or antigenic fragment thereof. In some embodiments, the antigenic protein comprises human metapneumovirus (hMPV) F protein or an antigenic fragment thereof. In some embodiments, the antigenic protein comprises an amino acid sequence having at least about 80%, about 85%, about 90%, about 91%, about 92%, about 93%, about 94%, about 95%, about 96%, about 97%, about 98%, about 99%, or about 100% identity to an amino acid sequence selected from selected from SEQ ID NOs: 64-136 and 206-209. In some embodiments, the antigenic protein comprises a polypeptide selected from SEQ ID NOs: 64-136 and 206-209.

In some embodiments, the fusion protein comprises a peptide linker positioned between the compA protein and the antigenic protein. In some embodiments, the peptide linker is a Gly-Ser linker. In some embodiments, the Gly-Ser linker comprises an amino acid sequence selected from SEQ ID NOs: 213-215. In some embodiments, the Gly-Ser linker consists of an amino acid sequence selected from SEQ ID NOs: 213-215.

In some embodiments, the fusion protein comprises an amino acid sequence having at least about 80%, about 85%, about 90%, about 91%, about 92%, about 93%, about 94%, about 95%, about 96%, about 97%, about 98%, about 99%, or about 100% identity to an amino acid sequence selected from SEQ ID NOs: 61 and 167-193. In some embodiments, the fusion protein comprises an amino acid sequence having at least about 80%, about 85%, about 90%, about 91%, about 92%, about 93%, about 94%, about 95%, about 96%, about 97%, about 98%, about 99%, or about 100% identity to an amino acid sequence selected from SEQ ID NOs: 60, 137-166, and 194-199. In some embodiments, the fusion protein comprises an amino acid sequence having at least about 80%, about 85%, about 90%, about 91%, about 92%, about 93%, about 94%, about 95%, about 96%, about 97%, about 98%, about 99%, or about 100% identity to an amino acid sequence selected from SEQ ID NOs: 200-205.

In some embodiments, the disclosure provides a method of making a nanostructure, comprising adding a fusion protein to a solution comprising a component B (compB) protein under conditions that minimize shear stress, wherein the fusion protein comprises a compA protein linked to an antigenic protein, and wherein the fusion protein comprises an amino acid sequence having at least about 80%, about 85%, about 90%, about 91%, about 92%, about 93%, about 94%, about 95%, about 96%, about 97%, about 98%, about 99%, or about 100% identity to a polypeptide selected from SEQ ID NOs: 137-205, thereby forming a compA:compB complex.

In some embodiments, the method provides a complex having icosahedral symmetry.

In some aspects, the disclosure provides a complex produced by a method described herein.

In some aspects, the disclosure provides a pharmaceutical composition comprising a complex produced by a method described herein, and a pharmaceutically acceptable diluent.

In some aspects, the disclosure provides a vaccine comprising a complex produced by a method described herein.

In some aspects, the disclosure provides a method of treating or preventing a disease or disorder in a subject in need thereof, comprising administering an effective amount of a complex described herein, a pharmaceutical composition described herein, or a vaccine described herein to the subject. In some embodiments, the disease or disorder is a viral infection.

In some aspects, the disclosure provides a method of generating an immune response in a subject in need thereof, comprising administering an effective amount of a complex described herein, a pharmaceutical composition described herein, or a vaccine described herein to the subject.

In some aspects, the disclosure provides a kit comprising a complex described herein, a pharmaceutical composition described herein, or a vaccine described herein.

In some aspects, the disclosure provides a complex described herein, a pharmaceutical composition described herein, or a vaccine described herein for use as a medicament. In some embodiments, the disclosure provides a complex described herein, a pharmaceutical composition described herein, or a vaccine described herein, for use in treating or preventing a disease or disorder in a subject in need thereof.

Further aspects and embodiments of the invention will be evident from the detailed description that follows.

### BRIEF DESCRIPTION OF THE DRAWINGS

FIG. 1A shows an illustrative embodiment of a protein-based virus-like particle (pbVLP) according to the present disclosure. The pbVLP is formed from (i) an antigen fused to a component A (compA) protein described herein that forms a trimer; and (ii) a component B (compB) protein described herein that forms a pentamer. The assembly of compA and compB proteins results in pbVLP formation.

FIG. 1B shows further illustrative embodiments of VLPs and VLP components (with G protein not shown).

FIG. 2 shows a nearest-neighbor joining tree of compA and compB proteins.

FIG. 3A shows the configuration of needle containing compA and tube containing compB used for dropwise addition. Mechanical mixing was performed with a stir bar.

FIG. 3B shows turbid solutions generated by dropwise addition. The arrow indicates presence of protein precipitate in the mixture.

FIG. 4 shows an HPLC analysis of assembled pbVLP. The pbVLP were prepared by addition of a solution of compA under a solution of compB using a pipette and mixing using an orbital shaker. Analysis by size-exclusion chromatography (SEC) showed presence of assembled pbVLP with a small peak due to high molecular weight (HMW) aggregate, presence of unassembled CompA protein, and impurities.

FIGS. 5A-5D show HPLC analysis of assembled pbVLP before or after purification by tangential flow filtration (TFF). FIG. 5A shows SEC-HPLC analysis of a crude pbVLP mixture prior to purification. FIGS. 5B-5D shows SEC-HPLC analysis of pbVLP following TFF.

FIG. 6 shows chromatographic purification with Captocore700. The SEC-HPLC profile of the VLP population changed significantly

FIG. 7 shows SEC-HPLC analysis of assembled pbVLP following chromatographic purification where both the Pellicon Biomax 1000 kDa membrane and Captocore were used in sequence. “Load TFF” represents the pbVLP mixture prior to filtration, “TFF Ultracel 1000 kDa” represents the pbVLP mixture following TFF, and “Captocore flowthrough” represents the pbVLP mixture following TFF then Captocore. The experiment showed that membrane and Captocore purification were redundant. Captocore impacted the SEC-HPLC profile (HMW peak 13.6% of VLP following filtration and 20.4% of VLP following Captocore). The bottom panel provides an overlay of the peaks.

FIG. 8A shows SEC-HPLC analysis for pbVLP assembled following addition of a trimer of CompA fused to human MPV antigen to a CompB pentamer with mixing performed using a pipette or an Ignite microfluidic mixing system. Shown is analysis of unassembled CompA-hMPV trimer (R1), CompA-hMPV/CompB mixed by pipette (R2), or CompA-hMPV/CompB mixed by the Ignite microfluidic mixing system using a flow rate of 2 mL/min or 10 mL/min (R3 and R4 respectively).

FIG. 8B shows SEC-HPLC analysis of pbVLP assembled following addition of a trimer of CompA fused to human RSV antigen to a CompB pentamer with mixing performed using a pipette or an Ignite microfluidic mixing system. Shown is analysis of unassembled CompA-RSV trimer (R9), CompA-RSV/CompB mixed by pipette (R10), or CompA-RSV/CompB mixed by the Ignite microfluidic mixing system using a flow rate of 2 mL/min or 10 mL/min (R11 and R12 respectively).

FIGS. 9A-9B show SEC-HPLC analysis of pbVLP assembled following addition of a trimer of CompA fused to human RSV antigen (FIG. 9A) or human MPV antigen (FIG. 9B) to a CompB pentamer with mixing performed using a pipette. Comparison is made to a standard containing AAV capsid, a set of protein standards, or unassembled CompA.

### DETAILED DESCRIPTION OF THE INVENTION

The present disclosure demonstrates manufacturing of a two-component protein-based Virus-Like Particle (bpVLP) by continuous additional of a component A (compA) protein to a solution of a component B (compB) protein. Advantageously, the compA protein is mixed with the compB protein by slow adding to the compB under conditions that minimize shear stress (e.g., addition under the surface of the compB solution and/or additional without mechanical stirring). The compA:compB assembly may be purified from excess compA by filtration against a 1000 kDa membrane or the equivalent. Optionally the membrane is a regenerated cellulose membrane (e.g., a Pellicon® Ultracel® 1000 kDa membrane or the equivalent).

### Definitions

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

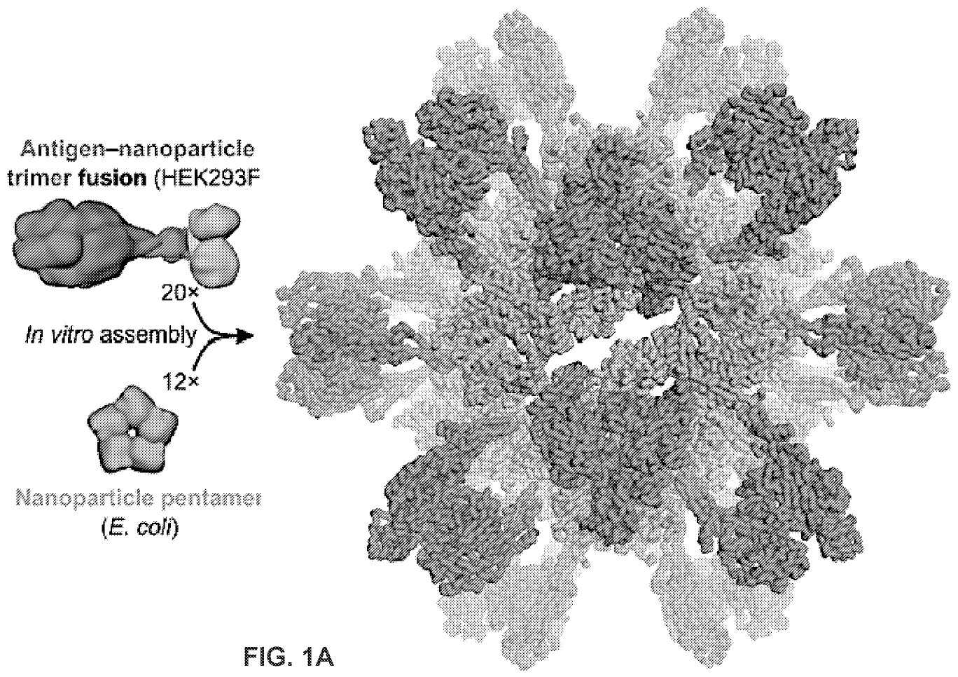

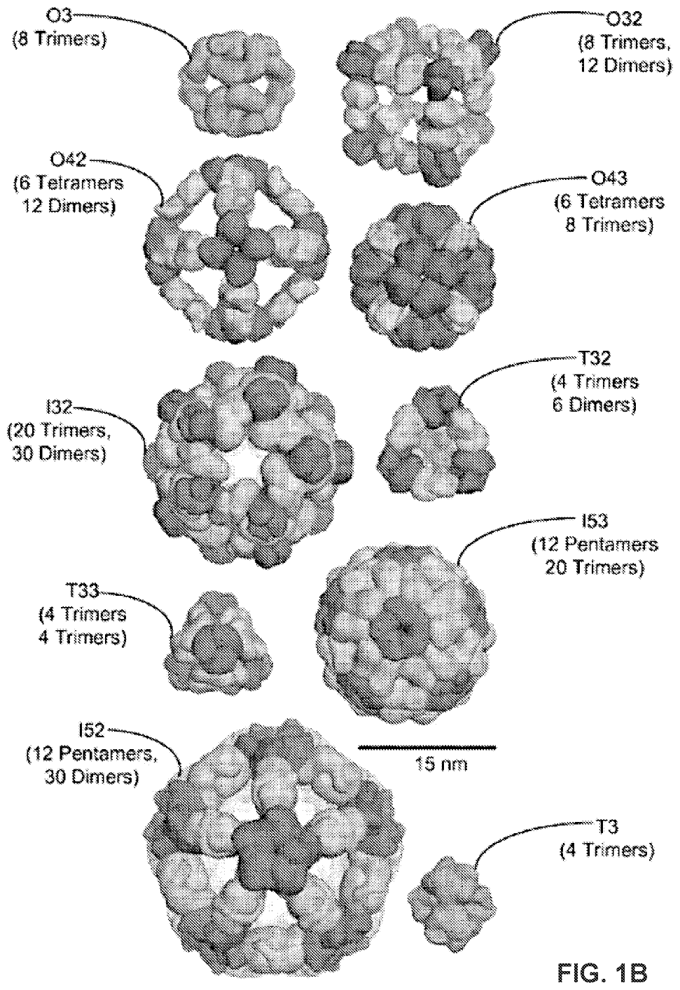

The term “virus-like particle” or “VLP” refers to a molecular assembly that resembles a virus but is non-infectious that displays an antigenic protein, or antigenic fragment thereof, of a viral protein or glycoprotein. A “protein-based VLP” refers to a VLP formed from proteins or glycoproteins and substantially free of other components (e.g., lipids). Protein-based VLPs may include post-translation modification and chemical modification, but are to be distinguished from micellar VLPs and VLPs formed by extraction of viral proteins from live or live inactivated virus preparations. The term “designed VLP” refers to a VLP comprising one or more polypeptides generated by computational protein design. Illustrative designed VLP are VLPs that comprise nanostructures depicted in FIG. 1B. The term “symmetric VLP” refers to a protein-based VLP with a symmetric core, such as shown in FIG. 1B. These include but are not limited to designed VLPs. For example, the protein ferritin has been used to generate a symmetric, protein-based VLP using naturally occurring ferritin sequences. Ferritin-based VLPs are distinguished from designed VLPs in that no protein engineering is necessary to form a symmetric VLP from ferritin, other than fusing the viral protein to the ferritin molecule. Protein design methods can be used to generate similar one- and two-component nanostructures based on template structures (e.g., structures deposited in the Protein Data Bank) or de novo (i.e., by computational design of new proteins having a desired structure but little or no homology to naturally occurring proteins). Such one- and two-component nanostructures can then be used as the core of a designed VLP.

The term “icosahedral particle” refers to a designed VLP having a core with icosahedral symmetry (e.g., the particles labeled 153 and 152 in Table 1). 153 refers to an icosahedral particle constructed from pentamers and trimers. 152 refers to an icosahedral particle constructed from pentamers and dimers. T33 refers to a tetrahedral particle constructed from two sets of trimers. T32 refers to a tetrahedral particle constructed from trimers and dimers.

The term “polypeptide” refers to a series of amino acid residues joined by peptide bonds and optionally one or more post-translational modifications (e.g., glycosylation) and/or other modifications (including but not limited to conjugation of the polypeptide moiety used as a marker—such as a fluorescent tag—or an adjuvant).

The term “variant” refers to a polypeptide having one or more insertions, deletions, or amino acid substitutions relative to a reference polypeptide, but retains one or more properties of the reference protein.

The term “functional variant” refers to a variant that exhibits the same or similar functional effect(s) as a reference polypeptide. For example, a functional variant of a multimerization domain is able to promote multimerization to the same extent, or to similar extent, as a reference multimerization domain and/or is able to multimerize with the same cognate multimerization domains as a reference multimerization domain.

The term “fragment” refers to a polypeptide having one or more N-terminal or C-terminal truncations compared to a reference polypeptide.

The term “functional fragment” refers to a functional variant of a fragment.

The term “amino acid substitution” refers to replacing a single amino acid in a sequence with another amino acid residue. The standard form of abbreviations for amino acid substitution are used. For example, V94R refers to substitution of valine (V) in a reference sequence with arginine (R). The abbreviation Arg94 refers to any sequence in which the 94^{th} residue, relative to a reference sequence, is arginine (Arg).

The terms “helix” or “helical” refer to an α-helical secondary structure in a polypeptide that is known to occur, or predicted to occur. For example, a sequence may be described as helical when computational modeling suggests the sequence is likely to adopt a helical conformation.

The term “component” refers to a protein, or protein complex, capable of assembly into a virus-like particle under appropriate conditions (e.g., an antigen or polypeptide comprising a multimerization domain). “Component A” or “compA” and “Component B” or “CompB” refer to two proteins capable of assembling to form a pbVLP as described herein. CompA and CompB are capable of independently forming dimer, trimer, or pentamer structures as described herein for use in assembly of the pbVLP. In some embodiments, compA is linked to an antigen to form a fusion protein.

The term “pharmaceutically acceptable excipients” means excipients biologically or pharmacologically compatible for in vivo use in animals or humans, and can mean excipients approved by a regulatory agency of the Federal or a state government or listed in the U.S. Pharmacopeia or other generally recognized pharmacopeia for use in animals, and more particularly in humans.

The term “manufacturing” refers to production of a recombinant polypeptide or virus-like particle at any scale, including at least 25-mL, 50-mL, 1-L, 1,000-L, 50,000-L, or greater scale.

The terms “culturing” and “culture medium” refers to standard cell culture and recombinant protein expression techniques.

The term “host cell” refers to any cell capable of use in expression of a recombinant polypeptide.

The term “mixing” refers to placing two solutions into contact to permit the solutions to mix.

The term “purify” refers to separating a molecule from other substances present in a composition. Polypeptides may be purified by affinity (e.g., to an antibody or to a tag, e.g., using a His-tag capture resin), by charge (e.g., ion-exchange chromatography), by size (e.g., preparative ultracentrifugation, size exclusion chromatography), or otherwise.

The terms “polynucleotide” and “nucleic acid,” used interchangeably herein, refer to a polymeric form of nucleotides of more than about 100 nucleotides, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The terms “identity”, “identical”, and “sequence identity” refer to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

Methods of sequence alignment for comparison and determination of percent sequence identity is well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of up to 20%, up to 10%, or up to 5%.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

As used herein, the term “denature” refers to a change in the structure of a folded polypeptide molecule that causes the polypeptide to lose all or substantially all tertiary structure, or in the case of a misfolded protein, to convert from an aggregated form into a soluble, unfolded form. The term “denatured” also refers a biologically inactive form of the expressed protein, as obtained as a product of the recombinant production process, after solubilizing inclusion bodies under conditions under which the native three-dimensional structure of the protein is disrupted.

The term “refolding” (or “renaturing”) refers to a process that causes a denatured protein to regain its native conformation and biological activity.

As used herein, the term recovering refers to obtaining of a substance (e.g. inclusion bodies and/or a protein of interest) by separating the substance from other substances in a preparation, e.g., by centrifugation and/or one or more wash steps.

As used herein, the term “inclusion body” refers to insoluble aggregates containing recombinant protein present in the cytoplasm of transformed host cells. These appear as bright spots under the microscope and can be recovered by separation of inclusion bodies from the cytoplasm of the cell. In the prior art, inclusion bodies are typically solubilized using high concentrations of a chaotropic agent (e.g. >8 M urea and/or >3 M guanidinium); a strong ionic detergent (e.g., N-lauroylsarcosine); and/or alkaline pH. According to the present disclosure, lower concentrations of these agents may be used to gently solubilize

As used herein, the term “nanostructure” include symmetrically repeated, non-natural, non-covalent protein-protein interfaces that orient a first component molecule (e.g. compA) and a second component molecule (e.g. compB) into a structure. Nanostructures include, but are not limited to, delivery vehicles, as the nanostructures can encapsulate molecules of interest and/or the first and/or second proteins can be modified to bind to molecules of interest (diagnostics, therapeutics, detectable molecules for imaging and other applications, etc.). The nanostructures of the disclosure are well suited for several applications, including vaccine design, targeted delivery of therapeutics, and bioenergy.

As used herein the term “solubilization” refers to a transfer of proteins comprised within a biological sample to a solvent such as an aqueous solvent by disrupting the cells of the biological sample. As used herein, “solubilization” or “solubilize” may be used interchangeably with “to dissolve” or “to extract”. The term “solubilization” also refers to the release a protein from inclusion bodies, e.g., by dissolving the inclusion bodies.

The following description includes information that may be useful in understanding the present invention. It is not an admission that any of the information provided herein is prior art or relevant to the presently claimed inventions, or that any publication specifically or implicitly referenced is prior art.

### EMBODIMENTS

Manufacturing capacity is determined by several factors. The amount of time required to make and release a batch (the run rate) is one of those factors. A continuous approach to manufacturing can reduce batch manufacturing and release time considerably thus increasing the capacity of a given facility or new investment. As disclosure herein, component flow rates may be controlled to maintain proper order of addition and molar ratios of the components. In the methods of the disclosure, compA protein is mixed with pre-manufactured compB. The compA protein may be provided in excess to the compB protein. Optionally, compA protein is added to the compB protein as the compA protein emerges from the last step in its purification process. Mixing of the components may be performed by static mixing elements or the equivalent. In some cases, the mixed components are delivered directly from the continuous assembly reaction to a 1000 kDa membrane processing unit. There may, in some cases, be a dwell time prior to contacting the assembly to the 1000 kDa membrane. The unit may operate in single-pass tangential flow filtration (TFF) mode. Advantageously, the method yields a steady stream of formulated drug substance (DS) ready for frozen storage. This approach may obviate the compA TFF formulation step and several vessels and hold steps in the compA process prior to pbVLP assembly. Advantageously, compA batch release and associated activities may not be necessary.

In some embodiments, compA may also be manufactured via continuous processing. Continuous compA manufacturing and assembly processes may be integrated into one continuous process running from bioreactor harvest directly to finished pbVLP DS.

In some embodiments, one integrated compA-to-DS continuous processing line is sized to match with about six bioreactors. The bioreactors would be harvested in a serial fashion. For example, a bioreactor is harvested and a drug substance is produced three days later (˜60 hr cycle) just as the next bioreactor is ready to harvest.

Protein-Based Virus Like Particle (pbVLP)

Vaccination is a treatment modality used to prevent or decrease the severity of infection with various infectious agents, including bacteria, viruses, and parasites. Development of new vaccines has important commercial and public health implications. In particular, lyme disease, pertussis, herpes virus, orthomyxovirus, paramyxovirus, pneumovirus, filovirus, flavivirus, reovirus, retrovirus, coronovirus, and malaria are infectious agents for which vaccines already exist, are being developed, or would be desirable.

Subunit vaccines are vaccines made from isolated antigens, usually proteins expressed recombinantly in bacterial, insect, or mammalian cell hosts. Typically, the antigenic component of a subunit vaccine is selected from among the proteins of an infectious agent observed to elicit a natural immune response upon infection, although in some cases other components of the infectious agent can be used. Typical antigens for use in subunit vaccines include protein expressed on the surface of the target infectious agent, as such surface-expressed envelope glycoproteins of viruses. Preferably, the antigen is a target for neutralizing antibodies. More preferably, the antigen is a target for broadly neutralizing antibodies, such that the immune response to the antigen covers immunity against multiple strains of the infectious agent. In some cases, glycans that are N-linked or O-linked to the subunit vaccine may also be important in vaccination, either by contributing to the epitope of the antigen or by guiding the immune response to particular epitopes on the antigen by steric hindrance. The immune response that occurs in response to vaccination may be direct to the protein itself, to the glycan, or to both the protein and linked glycans. Subunit vaccines have various advantages including that they contain no live pathogen, which eliminates concerns about infection of the patient by the vaccine; they may be designed using standard genetic engineering techniques; they are more homogenous than other forms of vaccine; and they can be manufactured in standardized recombinant protein expression production systems using well-characterized expression systems. In some cases, the antigen may be genetically engineered to favor generation of desirable antibodies, such as neutralizing or broadly neutralizing antibodies. In particular, structural information about an antigen of interest, obtained by X-ray crystallography, electron microscopy, or nuclear magnetic resonance experiments, can be used to guide rational design of subunit vaccines.

A known limitation of subunit vaccines is that the immune response elicited may sometimes be weaker than the immune response to other types of vaccines, such as whole virus, live, or live-attenuated vaccines. Designed and/or protein-based VLP vaccines have the potential to harness the advantages of subunit vaccines while increasing the potency and breadth of the vaccine-induced immune response through multivalent display of the antigen in symmetrically ordered arrays. In the present disclosure, protein-based VLPs are distinguished from nanoparticle vaccines, because the term nanoparticle vaccine has been used in the art to refer to protein-based or glycoprotein-based vaccines (see, e.g. U.S. Pat. No. 9,441,019), polymerized liposomes (see, e.g., U.S. Pat. No. 7,285,289), surfactant micelles (see, e.g., US Patent Pub. No. US 2004/0038406 A1), and synthetic biodegradable particles (see, e.g., U.S. Pat. No. 8,323,696).

A non-limiting example of an embodiment is shown in FIG. 1A, which depicts a protein antigen genetically fused to a component A (compA) protein of the pbVLP, which optionally is expressed recombinantly in a host cell (e.g., 293F cells); along with a component B (compB) protein assembly, which is expressed recombinantly in a host cell (e.g., E. coli cells), these two components self-assembling into a pbVLP displaying 20 copies of the protein antigen around an icosahedral core.

In some embodiments, compA is a dimer. In some embodiments, compA is a trimer. In some embodiments, compA is a pentamer.

In some embodiments, compB is a dimer. In some embodiments, compB is a trimer. In some embodiments, compA is a pentamer.

In some embodiments, compA is a dimer selected from SEQ ID Nos: 13,17, or 41. In some embodiments, compA is a trimer selected from SEQ ID Nos: 5, 7, 9, 19, 21, 25, 26, 29, 30, 31, 37, 39, 43, 45, 47, 49, or 51. In some embodiments, compA is a pentamer selected from SEQ ID Nos: 3, 11, 15, 23, or 35.

In some embodiments, compB is a dimer selected from SEQ ID Nos: 12, 16, 20, or 22. In some embodiments, compB is a trimer selected from SEQ ID Nos: 4, 18, 24, 34, 36, 42, 44, 46, 48, or 50. In some embodiments, compB is a pentamer selected from SEQ ID Nos: 2, 6, 8, 10, 14, 27, 28, 32, 33, 38, or 40.

In some embodiments, compA comprises a polypeptide sequence that has at least 90%, at least 95%, at least at least 99%, or 100% identity to any one of SEQ IN NOs: 1, 3, 5, 7, 9, 11, 13, 15, 17, 19, 21, 23, 25, 26, 29-31, 35, 37, 39, 41, 43, 45, 47, 49, 51, 53, 55, 57, and 59.

In some embodiments, compB comprises a polypeptide sequence that has at least 90%, at least 95%, at least 99%, or 100% identity to any one of SEQ IN NOs: 2, 4, 6, 8, 10, 12, 14, 16, 18, 20, 22, 24, 27, 28, 32-34, 36, 38, 40, 42, 44, 46, 48, 50, 52, 54, 56, and 58.

In some embodiments, compA and Comp B form an “153” architecture. An 153 architecture is a combination of 12 pentameric building blocks and twenty trimeric building blocks aligned along the five-fold and three-fold icosahedral symmetry axes as described in Bale et al., Science 353:389-394 (2016). In some embodiments, compA is the 153 pentamer. In some embodiments, compA is the 153 trimer. In some embodiments, compB is the 153 pentamer. In some embodiments, compB is the 153 trimer.

In some embodiments, compA and compB form an “152” architecture. An 152 architecture is formed from twelve pentamers and thirty dimers along their corresponding icosahedral symmetry axes. In some embodiments, compA is the 152 pentamer. In some embodiments, compA is the 152 dimer. In some embodiments, compB is the 152 pentamer. In some embodiments, compB is the 152 dimer.

In some embodiments, compA and compB form an “132” architecture. An 132 architecture is a combination of twenty trimers and thirty dimers, each aligned along their corresponding icosahedral symmetry axes. In some embodiments, compA is the 132 trimer. In some embodiments, compA is the 132 dimer. In some embodiments, compB is the 132 trimer. In some embodiments, compB is the 132 dimer.

In some embodiments, a mixture of compA and compB forms an icosahedral nanostructure. In some embodiments, a mixture of compA and compB forms a tetrahedral nanostructure. In some embodiments, a mixture of compA and compB forms an octahedral nanostructure.

In some embodiments, a small-molecule drug (i.e., with MW of less than 700), biological drug (i.e., drugs isolated from a bacterium, yeast, cell, or organ, especially including recombinant polypeptides), or biosynthetic drugs (e.g., aptamers, antisense nucleic acid, siRNA, recombinant nucleic acid, nucleoside analogs, recombinant polypeptides, polypeptide drugs, antigens, etc) is fused to compA.

In some embodiments, an antigen is fused to compA

In some embodiments, compA and compB form a nanostructure.

In some embodiments, the nanostructure is formed by combining a compA selected from one of SEQ ID NOs: 1, 3, 5, 7, 9, 11, 13, 15, 17, 19, 21, 23, 25, 26, 29-31, 35, 37, 39, 41, 43, 45, 47, 49, 51, 53, 55, 57, and 59, and a compB selected from one of SEQ ID NOs: 2, 4, 6, 8, 10, 12, 14, 16, 18, 20, 22, 24, 27, 28, 32-34, 36, 38, 40, 42, 44, 46, 48, 50, 52, 54, 56, and 58.

In some embodiments, the nanostructure is formed from a compA and compB polypeptide sequence that has at least 90%, at least 95%, at least 99%, or 100% identity to any one of:
(i) SEQ ID NO:1 and SEQ ID NO:2 respectively;
(ii) SEQ ID NO:3 and SEQ ID NO:4 respectively;
(iii) SEQ ID NO:3 and SEQ ID NO:24 respectively;
(iv) SEQ ID NO:23 and SEQ ID NO:4 respectively;
(v) SEQ ID NO:35 and SEQ ID NO:36 respectively;
(vi) SEQ ID NO:5 and SEQ ID NO:6 respectively;
(vii) SEQ ID NO:5 and SEQ ID NO:27 respectively;
(viii) SEQ ID NO:5 and SEQ ID NO:28 respectively;
(ix) SEQ ID NO:25 and SEQ ID NO:6 respectively;
(x) SEQ ID NO:25 and SEQ ID NO:27 respectively;
(xi) SEQ ID NO:25 and SEQ ID NO:28 respectively;
(xii) SEQ ID NO:26 and SEQ ID NO:6 respectively;
(xiii) SEQ ID NO:26 and SEQ ID NO:27 respectively;
(xiv) SEQ ID NO:26 and SEQ ID NO:28 respectively;
(xv) SEQ ID NO:37 and SEQ ID NO:38 respectively;
(xvi) SEQ ID NO:7 and SEQ ID NO:8 respectively;
(xvii) SEQ ID NO:7 and SEQ ID NO:32 respectively;
(xviii) SEQ ID NO:7 and SEQ ID NO:33 respectively;
(xix) SEQ ID NO:7 and SEQ ID NO:34 respectively;
(xx) SEQ ID NO:29 and SEQ ID NO:8 respectively;
(xxi) SEQ ID NO:29 and SEQ ID NO:32 respectively;
(xxii) SEQ ID NO:29 and SEQ ID NO:33 respectively;
(xxiii) SEQ ID NO:29 and SEQ ID NO:34 respectively;
(xxiv) SEQ ID NO:30 and SEQ ID NO:8 respectively;
(xxv) SEQ ID NO:30 and SEQ ID NO:32 respectively;
(xxvi) SEQ ID NO:30 and SEQ ID NO:33 respectively;
(xxvii) SEQ ID NO:30 and SEQ ID NO:34 respectively;
(xxviii) SEQ ID NO:31 and SEQ ID NO:8 respectively;
(xxix) SEQ ID NO:31 and SEQ ID NO:32 respectively;
(xxx) SEQ ID NO:31 and SEQ ID NO:33 respectively;
(xxxi) SEQ ID NO:31 and SEQ ID NO:34 respectively;
(xxxii) SEQ ID NO:39 and SEQ ID NO:40 respectively;
(xxxiii) SEQ ID NO:9 and SEQ ID NO:10 respectively;
(xxxiv) SEQ ID NO:11 and SEQ ID NO:12 respectively;
(xxxv) SEQ ID NO:13 and SEQ ID NO:14 respectively;
(xxxvi) SEQ ID NO:15 and SEQ ID NO:16 respectively;
(xxxvii) SEQ ID NO:19 and SEQ ID NO:20 respectively;
(xxxviii) SEQ ID NO:21 and SEQ ID NO:22 respectively;
(xxxix) SEQ ID NO:23 and SEQ ID NO:24 respectively;
(xl) SEQ ID NO:41 and SEQ ID NO:42 respectively;
(xli) SEQ ID NO:43 and SEQ ID NO:44 respectively;
(xlii) SEQ ID NO:45 and SEQ ID NO:46 respectively;
(xliii) SEQ ID NO:47 and SEQ ID NO:48 respectively;
(xliv) SEQ ID NO:49 and SEQ ID NO:50 respectively;
(xlv) SEQ ID NO:51 and SEQ ID NO:44 respectively;
(xlvi) SEQ ID NO:53 and SEQ ID NO:52 respectively;
(xlvii) SEQ ID NO:55 and SEQ ID NO:54 respectively;
(xlviii) SEQ ID NO:57 and SEQ ID NO:56 respectively; and
(xlix) SEQ ID NO:59 and SEQ ID NO:58 respectively.

In some embodiments, compA comprises a helical extension. In some embodiments, the helical extension is located at the N-terminus of compA. In some embodiments, the helical extension is EKAAKAEEAARK (SEQ ID NO: 62).

In some embodiments, compB comprises a helical extension. In some embodiments, the helical extension is located at the N-terminus of compB. In some embodiments, the helical extension is EKAAKAEEAARK (SEQ ID NO: 62).

In some embodiments, the nanostructure is a pbVLP. In some embodiments, the pbVLP is a vaccine.

Other compA/compB assemblies of the present disclosure are shown in FIG. 1B. In some embodiments, the pbVLP is adapted for display of up to 8 trimers; 8 trimers and 12 dimers; 6 tetramers and 12 dimers; 6 tetramers and 8 trimers; 20 trimers and 30 dimers; 4 trimers and 6 dimers; 4 first trimers and 4 second trimers, or 8 trimers; 12 pentamers and 20 trimers; or 12 pentamers and 30 dimers; or 4 trimers. In some cases, one of the symmetric axes is not used for antigen display, thus, in some embodiments the pbVLP is adapted for display of up to 8 trimers; 12 dimers; 6 tetramers; 20 trimers; 30 dimers; 4 trimers; 6 dimers; 8 trimers; or 12 pentamers. In some cases, monomeric antigens are displayed and thus, the pbVLP is adapted for display of up to 12, 24, 60, or 70 monomeric antigens. In some cases, the pbVLP comprises mixed pluralities of polypeptides such that otherwise identical polypeptides of the core of the pbVLP display different antigens or no antigen. Thus, depending on the ratio of polypeptides, the pbVLP is in some cases adapted for display of between 1 and 130 antigens (e.g., on the 152 particle) where each of the antigens displayed may be the same or may be different members of mixed population in proportion to any ratio chosen. The antigens may be co-expressed in a recombinant expression system and self-assembled before purification. Non-limiting exemplary pbVLPs are provided in Bale et al. Science 353:389-94 (2016); Heinze et al. J. Phys. Chem B. 120:5945-5952 (2016); King et al. Nature 510:103-108 (2014); and King et al. Science 336:1171-71 (2012).

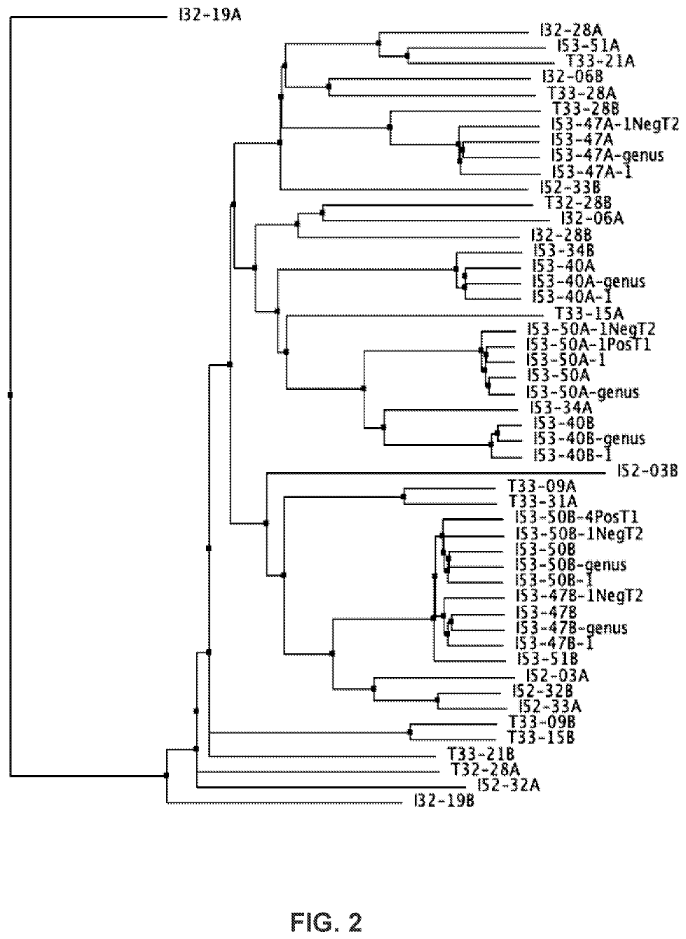

The compA and compB proteins of the present disclosure may have any of various amino acid sequences. U.S. Patent Pub No. US 2015/0356240 A1 describes various methods for designing protein assemblies. As described in US Patent Pub No. US 2016/0122392 A1 and in International Patent Pub. No. WO 2014/124301 A1, the isolated polypeptides of SEQ ID NOS:1-51 were designed for their ability to self-assemble in pairs to form pbVLPs, such as icosahedral particles. The design involved design of suitable interface residues for each member of the polypeptide pair that can be assembled to form the pbVLP. The pbVLPs so formed include symmetrically repeated, non-natural, non-covalent polypeptide-polypeptide interfaces that orient a first assembly and a second assembly into a pbVLP, such as one with an icosahedral symmetry. Thus, in one embodiment the compA and compBs (that is, the two polypeptides of the core of the VLP) are selected from the group consisting of SEQ ID NOS:1-51. In each case, an N-terminal methionine residue present in the full-length protein but typically removed to make a fusion is not included in the sequence. In various embodiments, one or more additional residues are deleted from the N-terminus and/or additional residues are added to the N-terminus (e.g. to form a helical extension). As shown in FIG. 2, the sequences disclosed below group into several families of related protein sequences.

TABLE 1

| | | | Identified |
|---|---|---|---|
| | Component | | interface |
| Name | Multimer | Amino Acid Sequence | residues |
| I53-34A | trimer | EGMDPLAVLAESRLLPLLTVRGGEDLAGLATVLELMGVGALEITLRTEKGLE | I53-34A: |
| SEQ ID NO: 1 | | ALKALRKSGLLLGAGTVRSPKEAEAALEAGAAFLVSPGLLEEVAALAQARGV | 28, 32, 36, 37, |
| | | PYLPGVLTPTEVERALALGLSALKFFPAEPFQGVRVLRAYAEVFPEVRFLPT | 186, 188, 191, |
| | | GGIKEEHLPHYAALPNLLAVGGSWLLQGDLAAVMKKVKAAKALLSPQAPG | 192, 195 |
| I53-34B | pentamer | TKKVGIVDTTFARVDMAEAAIRTLKALSPNIKIIRKTVPGIKDLPVACKKLL | I53-34B: |
| SEQ ID NO: 2 | | EEEGCDIVMALGMPGKAEKDKVCAHEASLGLMLAQLMTNKHIIEVFVHEDEA | 19, 20, 23, 24, |
| | | KDDDELDILALVRAIEHAANVYYLLFKPEYLTRMAGKGLRQGREDAGPARE | 27, 109, 113, |
| | | | 116, 117, 120, |
| | | | 124, 148 |
| I53-40A | pentamer | TKKVGIVDTTFARVDMASAAILTLKMESPNIKIIRKTVPGIKDLPVACKKLL | I53-40A: |
| SEQ ID NO: 3 | | EEEGCDIVMALGMPGKAEKDKVCAHEASLGLMLAQLMTNKHIIEVFVHEDEA | 20, 23, 24, 27, |
| | | KDDAELKILAARRAIEHALNVYYLLFKPEYLTRMAGKGLRQGFEDAGPARE | 28, 109, 112, |
| | | | 113, 116, 120, |
| | | | 124 |
| I53-40B | trimer | STINNQLKALKVIPVIAIDNAEDIIPLGKVLAENGLPAAEITFRSSAAVKAI | I53-40B: |
| SEQ ID NO: 4 | | MLLRSAQPEMLIGAGTILNGVQALAAKEAGATFVVSPGFNPNTVRACQIIGI | 47, 51, 54, 58, |
| | | DIVPGVNNPSTVEAALEMGLTTLKFFPAEASGGISMVKSLVGPYGDIRLMPT | 74, 102 |
| | | GGITPSNIDNYLAIPQVLACGGTWMVDKKLVTNGEWDEIARLTREIVEQVNP | |
| I53-47A | trimer | PIFTLNTNIKATDVPSDFLSLTSRLVGLILSKPGSYVAVHINTDQQLSFGGS | I53-47A: |
| SEQ ID NO: 5 | | TNPAAFGTLMSIGGIEPSKNRDHSAVLFDHLNAMLGIPKNRMYIHFVNLNGD | 22, 25, 29, 72, |
| | | DVGWNGTTF | 79, 86, 87 |
| I53-47B | pentamer | NQHSHKDYETVRIAVVRARWHADIVDACVEAFEIAMAAIGGDRFAVDVFDVP | I53-47B: |
| SEQ ID NO: 6 | | GAYEIPLHARTLAETGRYGAVLGTAFVVNGGIYRHEFVASAVIDGMMNVQLS | 28, 31, 35, 36, |
| | | TGVPVLSAVLTPHRYRDSAEHHRFFAAHFAVKGVEAARACIEILAAREKIAA | 39, 131, 132, |
| | | | 135, 139, 146 |
| I53-50A | trimer | MEELFKKHKIVAVLRANSVEEAIEKAVAVFAGGVHLIEITFTVPDADTVIKA | I53-50A: |
| SEQ ID NO: 7 | | LSVLKEKGAIIGAGTVTSVEQCRKAVESGAEFIVSPHLDEEISQFCKEKGVF | 25, 29, 33, 54, |
| | | YMPGVMTPTELVKAMKLGHTILKLFPGEVVGPQFVKAMKGPFPNVKFVPTGG | 57 |
| | | VNLDNVCEWFKAGVLAVGVGSALVKGTPDEVREKAKAFVEKIRGCTE | |
| I53-50B | pentamer | NQHSHKDYETVRIAVVRARWHAEIVDACVSAFEAAMADIGGDRFAVDVFDVP | I53-50B: |
| SEQ ID NO: 8 | | GAYEIPLHARTLAETGRYGAVLGTAFVVNGGIYRHEFVASAVIDGMMNVQLS | 24, 28, 36, 124, |
| | | TGVPVLSAVLTPHRYRDSDAHTLLFLALFAVKGMEAARACVEILAAREKIAA | 125, 127, 128, |
| | | | 129, |
| | | | 131, 132, 133, |
| | | | 135, 139 |
| I53-51A | trimer | FTKSGDDGNTNVINKRVGKDSPLVNFLGDLDELNSFIGFAISKIPWEDMKKD | I53-51A: |
| SEQ ID NO: 9 | | LERVQVELFEIGEDLSTQSSKKKIDESYVLWLLAATAIYRIESGPVKLFVIP | 80, 83, 86, 87, |
| | | GGSEEASVLHVTRSVARRVERNAVKYTKELPEINRMIIVYLNRLSSLLFAMA | 88, 90, 91, 94, |
| | | LVANKRRNQSEKIYEIGKSW | 166, 172, 176 |
| I53-51B | pentamer | NQHSHKDYETVRIAVVRARWHADIVDQCVRAFEEAMADAGGDRFAVDVFDVP | I53-51B: |
| SEQ ID | | GAYEIPLHARTLAETGRYGAVLGTAFVVNGGIYRHEFVASAVIDGMMNVQLS | 31, 35, 36, 40, |
| NO: 10 | | TGVPVLSAVLTPHRYRSSREHHEFFREHFMVKGVEAAAACITILAAREKIAA | 122, 124, 128, |
| | | | 131, 135, 139, |
| | | | 143, 146, 147 |
| I52-03A | pentamer | GHTKGPTPQQHDGSALRIGIVHARWNKTIIMPLLIGTIAKLLECGVKASNIV | I52-03A: |
| SEQ ID | | VQSVPGSWELPIAVQRLYSASQLQTPSSGPSLSAGDLLGSSTTDLTALPTTT | 28, 32, 36, 39, |
| NO: 11 | | ASSTGPFDALIAIGVLIKGETMHFEYIADSVSHGLMRVQLDTGVPVIFGVLT | 44, 49 |
| | | VLTDDQAKARAGVIEGSHNHGEDWGLAAVEMGVRRRDWAAGKTE | |
| I52-03B | dimer | YEVDHADVYDLFYLGRGKDYAAEASDIADLVRSRTPEASSLLDVACGTGTHL | I52-03B: |
| SEQ ID | | EHFTKEFGDTAGLELSEDMLTHARKRLPDATLHQGDMRDFQLGRKFSAVVSM | 94, 115, 116, |
| NO: 12 | | FSSVGYLKTVAELGAAVASFAEHLEPGGVVVVEPWWFPETFADGWVSADVVR | 206, 213 |
| | | RDGRTVARVSHSVREGNATRMEVHFTVADPGKGVRHFSDVHLITLFHQREYE | |
| | | AAFMAAGLRVEYLEGGPSGRGLFVGVPA | |
| I52-32A | dimer | GMKEKFVLIITHGDFGKGLLSGAEVIIGKQENVHTVGLNLGDNIEKVAKEVM | I52-32A: |
| SEQ ID | | RIIIAKLAEDKEIIIVVDLFGGSPFNIALEMMKTFDVKVITGINMPMLVELL | 47, 49, 53, 54, |
| NO: 13 | | TSINVYDTTELLENISKIGKDGIKVIEKSSLKM | 57, 58, 61, 83, |
| | | | 87, 88 |
| I52-32B | pentamer | KYDGSKLRIGILHARWNLEIIAALVAGAIKRLQEFGVKAENIIIETVPGSFE | I52-32B: |
| SEQ ID | | LPYGSKLFVEKQKRLGKPLDAIIPIGVLIKGSTMHFEYICDSTTHQLMKLNF | 19, 20, 23, 30, |
| NO: 14 | | ELGIPVIFGVLTCLTDEQAEARAGLIEGKMHNHGEDWGAAAVEMATKFN | 40 |
| I52-33A | pentamer | AVKGLGEVDQKYDGSKLRIGILHARWNRKIILALVAGAVLRLLEFGVKAENI | I52-33A: |
| SEQ ID | | IIETVPGSFELPYGSKLFVEKQKRLGKPLDAIIPIGVLIKGSTMHFEYICDS | 33, 41, 44, 50 |
| NO: 15 | | TTHQLMKLNFELGIPVIFGVLTCLTDEQAEARAGLIEGKMHNHGEDWGAAAV | |
| | | EMATKFN | |
| I52-33B | dimer | GANWYLDNESSRLSFTSTKNADIAEVHRFLVLHGKVDPKGLAEVEVETESIS | I52-33B: |
| SEQ ID | | TGIPLRDMLLRVLVFQVSKFPVAQINAQLDMRPINNLAPGAQLELRLPLTVS | 61, 63, 66, 67, |
| NO: 16 | | LRGKSHSYNAELLATRLDERRFQVVTLEPLVIHAQDFDMVRAFNALRLVAGL | 72, 147, 148, |
| | | SAVSLSVPVGAVLIFTAR | 154, 155 |
| I32-06A | dimer | TDYIRDGSAIKALSFAIILAEADLRHIPQDLQRLAVRVIHACGMVDVANDLA | I32-06A: |
| SEQ ID | | FSEGAGKAGRNALLAGAPILCDARMVAEGITRSRLPADNRVIYTLSDPSVPE | 9, 12, 13, 14, |
| NO: 17 | | LAKKIGNTRSAAALDLWLPHIEGSIVAIGNAPTALFRLFELLDAGAPKPALI | 20, 30, 33, 34 |
| | | IGMPVGFVGAAESKDELAANSRGVPYVIVRGRRGGSAMTAAAVNALASERE | |
| I32-06B | trimer | ITVFGLKSKLAPRREKLAEVIYSSLHLGLDIPKGKHAIRFLCLEKEDFYYPF | I32-06B: |
| SEQ ID | | DRSDDYTVIEINLMAGRSEETKMLLIFLLFIALERKLGIRAHDVEITIKEQP | 24, 71, 73, 76, |
| NO: 18 | | AHCWGFRGRTGDSARDLDYDIYV | 77, 80, 81, 84, |
| | | | 85, 88, 114, 118 |
| I32-19A | trimer | GSDLQKLQRFSTCDISDGLLNVYNIPTGGYFPNLTAISPPQNSSIVGTAYTV | I32-19A: |
| SEQ ID | | LFAPIDDPRPAVNYIDSVPPNSILVLALEPHLQSQFHPFIKITQAMYGGLMS | 208, 213, 218, |
| NO: 19 | | TRAQYLKSNGTVVFGRIRDVDEHRTLNHPVFAYGVGSCAPKAVVKAVGTNVQ | 222, 225, 226, |
| | | LKILTSDGVTQTICPGDYIAGDNNGIVRIPVQETDISKLVTYIEKSIEVDRL | 229, 233 |
| | | VSEAIKNGLPAKAAQTARRMVLKDYI | |
| I32-19B | dimer | SGMRVYLGADHAGYELKQAIIAFLKMTGHEPIDCGALRYDADDDYPAFCIAA | I32-19B: |
| SEQ ID | | ATRTVADPGSLGIVLGGSGNGEQIAANKVPGARCALAWSVQTAALAREHNNA | 20, 23, 24, 27, |
| NO: 20 | | QLIGIGGRMHTLEEALRIVKAFVTTPWSKAQRHQRRIDILAEYERTHEAPPV | 117, 118, 122, |
| | | PGAPA | 125 |
| I32-28A | trimer | GDDARIAAIGDVDELNSQIGVLLAEPLPDDVRAALSAIQHDLFDLGGELCIP | I32-28A: |
| SEQ ID | | GHAAITEDHLLRLALWLVHYNGQLPPLEEFILPGGARGAALAHVCRTVCRRA | 60, 61, 64, 67, |
| NO: 21 | | ERSIKALGASEPLNIAPAAYVNLLSDLLFVLARVLNRAAGGADVLWDRTRAH | 68, 71, 110, 120, |
| | | | 123, 124, 128 |
| I32-28B | dimer | ILSAEQSFTLRHPHGQAAALAFVREPAAALAGVQRLRGLDSDGEQVWGELLV | I32-28B: |
| SEQ ID | | RVPLLGEVDLPFRSEIVRTPQGAELRPLTLTGERAWVAVSGQATAAEGGEMA | 35, 36, 54, 122, |
| NO: 22 | | FAFQFQAHLATPEAEGEGGAAFEVMVQAAAGVTLLLVAMALPQGLAAGLPPA | 129, 137, 140, |
| | | | 141, 144, 148 |
| I53-40A.1 | pentamer | TKKVGIVDTTFARVDMASAAILTLKMESPNIKIIRKTVPGIKDLPVACKKLL | I53-40A: |
| SEQ ID | | EEEGCDIVMALGMPGKKEKDKVCAHEASLGLMLAQLMTNKHIIEVFVHEDEA | 20, 23, 24, 27, |
| NO: 23 | | KDDAELKILAARRAIEHALNVYYLLFKPEYLTRMAGKGLRQGFEDAGPARE | 28, 109, 112, |
| | | | 113, 116, 120, |
| | | | 124 |
| I53-40B.1 | trimer | DDINNQLKRLKVIPVIAIDNAEDIIPLGKVLAENGLPAAEITFRSSAAVKAI | I53-40B: |
| SEQ ID | | MLLRSAQPEMLIGAGTILNGVQALAAKEAGADFVVSPGFNPNTVRACQIIGI | 47, 51, 54, 58, |
| NO: 24 | | DIVPGVNNPSTVEQALEMGLTTLKFFPAEASGGISMVKSLVGPYGDIRLMPT | 74, 102 |
| | | GGITPDNIDNYLAIPQVLACGGTWMVDKKLVRNGEWDEIARLTREIVEQVNP | |
| I53-47A.1 | trimer | PIFTLNTNIKADDVPSDFLSLTSRLVGLILSKPGSYVAVHINTDQQLSFGGS | I53-47A: |
| SEQ ID | | TNPAAFGTLMSIGGIEPDKNRDHSAVLFDHLNAMLGIPKNRMYIHFVNLNGD | 22, 25, 29, 72, |
| NO: 25 | | DVGWNGTTF | 79, 86, 87 |
| I53- | trimer | PIFTLNTNIKADDVPSDFLSLTSRLVGLILSEPGSYVAVHINTDQQLSFGGS | I53-47A: |
| 47A.1NegT2 | | TNPAAFGTLMSIGGIEPDKNEDHSAVLFDHLNAMLGIPKNRMYIHFVDLDGD | 22, 25, 29, 72, |
| SEQ ID | | DVGWNGTTF | 79, 86, 87 |
| NO: 26 | | | |
| I53-47B.1 | pentamer | NQHSHKDHETVRIAVVRARWHADIVDACVEAFEIAMAAIGGDRFAVDVFDVP | I53-47B: |
| SEQ ID | | GAYEIPLHARTLAETGRYGAVLGTAFVVNGGIYRHEFVASAVIDGMMNVQLD | 28, 31, 35, 36, |
| NO: 27 | | TGVPVLSAVLTPHRYRDSDEHHRFFAAHFAVKGVEAARACIEILNAREKIAA | 39, 131, 132, |
| | | | 135, 139, 146 |
| I53- | pentamer | NQHSHKDHETVRIAVVRARWHADIVDACVEAFEIAMAAIGGDRFAVDVFDVP | I53-47B: |
| 47B.1NegT2 | | GAYEIPLHARTLAETGRYGAVLGTAFVVDGGIYDHEFVASAVIDGMMNVQLD | 28, 31, 35, 36, |
| SEQ ID | | TGVPVLSAVLTPHEYEDSDEDHEFFAAHFAVKGVEAARACIEILNAREKIAA | 39, 131, 132, |
| NO: 28 | | | 135, 139, 146 |
| I53-50A.1 | trimer | EELFKKHKIVAVLRANSVEEAIEKAVAVFAGGVHLIEITFTVPDADTVIKAL | I53-50A: |
| SEQ ID | | SVLKEKGAIIGAGTVTSVEQCRKAVESGAEFIVSPHLDEEISQFCKEKGVFY | 25, 29, 33, 54, |
| NO: 29 | | MPGVMTPTELVKAMKLGHDILKLFPGEVVGPQFVKAMKGPFPNVKFVPTGGV | 57 |
| | | NLDNVCEWFKAGVLAVGVGDALVKGDPDEVREKAKKFVEKIRGCTE | |
| I53- | trimer | EELFKKHKIVAVLRANSVEEAIEKAVAVFAGGVHLIEITFTVPDADTVIKAL | I53-50A: |
| 50A.1NegT2 | | SVLKEKGAIIGAGTVTSVEQCRKAVESGAEFIVSPHLDEEISQFCKEKGVFY | 25, 29, 33, 54, |
| SEQ ID | | MPGVMTPTELVKAMKLGHDILKLFPGEVVGPEFVEAMKGPFPNVKFVPTGGV | 57 |
| NO: 30 | | DLDDVCEWFDAGVLAVGVGDALVEGDPDEVREDAKEFVEEIRGCTE | |
| I53- | trimer | EELFKKHKIVAVLRANSVEEAIEKAVAVFAGGVHLIEITFTVPDADTVIKAL | I53-50A: |
| 50A.1PosT1 | | SVLKEKGAIIGAGTVTSVEQCRKAVESGAEFIVSPHLDEEISQFCKEKGVFY | 25, 29, 33, 54, |
| SEQ ID | | MPGVMTPTELVKAMKLGHDILKLFPGEVVGPQFVKAMKGPFPNVKFVPTGGV | 57 |
| NO: 31 | | NLDNVCKWFKAGVLAVGVGKALVKGKPDEVREKAKKFVKKIRGCTE | |
| I53-50B.1 | pentamer | NQHSHKDHETVRIAVVRARWHAEIVDACVSAFEAAMRDIGGDRFAVDVFDVP | I53-50B: |
| SEQ ID | | GAYEIPLHARTLAETGRYGAVLGTAFVVNGGIYRHEFVASAVIDGMMNVQLD | 24, 28, 36, 124, |
| NO: 32 | | TGVPVLSAVLTPHRYRDSDAHTLLFLALFAVKGMEAARACVEILAAREKIAA | 125, 127, 128, |
| | | | 129, 131, 132, |
| | | | 133, 135, 139 |
| I53- | pentamer | NQHSHKDHETVRIAVVRARWHAEIVDACVSAFEAAMRDIGGDRFAVDVFDVP | I53-50B: |
| 50B.1NegT2 | | GAYEIPLHARTLAETGRYGAVLGTAFVVDGGIYDHEFVASAVIDGMMNVQLD | 24, 28, 36, 124, |
| SEQ ID | | TGVPVLSAVLTPHEYEDSDADTLLFLALFAVKGMEAARACVEILAAREKIAA | 125, 127, 128, |
| NO: 33 | | | 129, 131, 132, |
| | | | 133, 135, 139 |
| I53- | trimer | NQHSHKDHETVRIAVVRARWHAEIVDACVSAFEAAMRDIGGDRFAVDVFDVP | I53-50B: |
| 50B.4PosT1 | | GAYEIPLHARTLAETGRYGAVLGTAFVVNGGIYRHEFVASAVINGMMNVQLN | 24, 28, 36, 124, |
| SEQ ID | | TGVPVLSAVLTPHNYDKSKAHTLLFLALFAVKGMEAARACVEILAAREKIAA | 125, 127, 128, |
| NO: 34 | | | 129, 131, 132, |
| | | | 133, 135, 139 |
| I53-40A | pentamer | TKKVGIVDTTFARVDMASAAILTLKMESPNIKIIRKTVPGIKDLPVACKKLL | |
| genus | | EEEGCDIVMALGMPGK(A/K)EKDKVCAHEASLGLMLAQLMTNKHIIEVFVH | |
| SEQ ID | | EDEAKDDAELKILAARRAIEHALNVYYLLFKPEYLTRMAGKGLRQGFEDAGP | |
| NO: 35 | | ARE | |
| I53-40B | trimer | (S/D)(T/D)INNQLK(A/R)LKVIPVIAIDNAEDIIPLGKVLAENGLPAAE | |
| genus | | ITFRSSAAVKAIMLLRSAQPEMLIGAGTILNGVQALAAKEAGA(T/D)FVVS | |
| SEQ ID | | PGFNPNTVRACQIIGIDIVPGVNNPSTVE(A/Q)ALEMGLTTLKFFPAEASG | |
| NO: 36 | | GISMVKSLVGPYGDIRLMPTGGITP(S/D)NIDNYLAIPQVLACGGTWMVDK | |
| | | KLV(T/R)NGEWDEIARLTREIVEQVNP | |
| I53-47A | trimer | PIFTLNTNIKA(T/D)DVPSDFLSLTSRLVGLILS(K/E)PGSYVAVHINTD | |
| genus | | QQLSFGGSTNPAAFGTLMSIGGIEP(S/D)KN(R/E)DHSAVLFDHLNAMLG | |
| SEQ ID | | IPKNRMYIHFV(N/D)L(N/D)GDDVGWNGTTF | |
| NO: 37 | | | |
| I53-47B | pentamer | NQHSHKD(Y/H)ETVRIAVVRARWHADIVDACVEAFEIAMAAIGGDRFAVDV | |
| genus | | FDVPGAYEIPLHARTLAETGRYGAVLGTAFVV(N/D)GGIY(R/D)HEFVAS | |
| SEQ ID | | AVIDGMMNVQL(S/D)TGVPVLSAVLTPH(R/E)Y(R/E)DS(A/D)E(H/D) | |
| NO: 38 | | H(R/E)FFAAHFAVKGVEAARACIEIL(A/N)AREKIAA | |
| I53-50A | trimer | EELFKKHKIVAVLRANSVEEAIEKAVAVFAGGVHLIEITFTVPDADTVIKAL | |
| genus | | SVLKEKGAIIGAGTVTSVEQCRKAVESGAEFIVSPHLDEEISQFCKEKGVFY | |
| SEQ ID | | MPGVMTPTELVKAMKLGH(T/D)ILKLFPGEVVGP(Q/E)FV(K/E)AMKGP | |
| NO: 39 | | FPNVKFVPTGGV(N/D)LD(N/D)VC(E/K)WF(K/D)AGVLAVGVG(S/K/ | |
| | | D)ALV(K/E)G(T/D/K)PDEVRE(K/D)AK(A/E/K)FV(E/K)(K/E)IR | |
| | | GCTE | |
| I53-50B | pentamer | NQHSHKD(Y/H)ETVRIAVVRARWHAEIVDACVSAFEAAM(A/R)DIGGDRF | |
| genus | | AVDVFDVPGAYEIPLHARTLAETGRYGAVLGTAFW(N/D)GGIY(R/D)HE | |
| SEQ ID | | FVASAVI(D/N)GMMNVQL(S/D/N)TGVPVLSAVLTPH(R/E/N)Y(R/D/ | |
| NO: 40 | | E)(D/K)S(D/K)A(H/D)TLLFLALFAVKGMEAARACVEILAAREKIAA | |
| T32-28A | dimer | GEVPIGDPKELNGMEIAAVYLQPIEMEPRGIDLAASLADIHLEADIHALKNN | |
| SEQ ID | | PNGFPEGFWMPYLTIAYALANADTGAIKTGTLMPMVADDGPHYGANIAMEKD | |
| NO: 41 | | KKGGFGVGTYALTFLISNPEKQGFGRHVDEETGVGKWFEPFVVTYFFKYTGT | |
| | | PK | |
| T32-28B | trimer | SQAIGILELTSIAKGMELGDAMLKSANVDLLVSKTISPGKFLLMLGGDIGAI | |
| SEQ ID | | QQAIETGTSQAGEMLVDSLVLANIHPSVLPAISGLNSVDKRQAVGIVETWSV | |
| NO: 42 | | AACISAADLAVKGSNVTLVRVHMAFGIGGKCYMVVAGDVLDVAAAVATASLA | |
| | | AGAKGLLVYASIIPRPHEAMWRQMVEG | |
| T33-09A | trimer | EEVVLITVPSALVAVKIAHALVEERLAACVNIVPGLTSIYRWQGSVVSDHEL | |
| SEQ ID | | LLLVKTTTHAFPKLKERVKALHPYTVPEIVALPIAEGNREYLDWLRENTG | |
| NO: 43 | | | |
| T33-09B | trimer | VRGIRGAITVEEDTPAAILAATIELLLKMLEANGIQSYEELAAVIFTVTEDL | |
| SEQ ID | | TSAFPAEAARLIGMHRVPLLSAREVPVPGSLPRVIRVLALWNTDTPQDRVRH | |
| NO: 44 | | VYLNEAVRLRPDLESAQ | |
| T33-15A | trimer | SKAKIGIVTVSDRASAGITADISGKAIILALNLYLTSEWEPIYQVIPDEQDV | |
| SEQ ID | | IETTLIKMADEQDCCLIVTTGGTGPAKRDVTPEATEAVCDRMMPGFGELMRA | |
| NO: 45 | | ESLKEVPTAILSRQTAGLRGDSLIVNLPGDPASISDCLLAVFPAIPYCIDLM | |
| | | EGPYLECNEAMIKPFRPKAK | |
| T33-15B | trimer | VRGIRGAITVNSDTPTSIIIATILLLEKMLEANGIQSYEELAAVIFTVTEDL | |
| SEQ ID | | TSAFPAEAARQIGMHRVPLLSAREVPVPGSLPRVIRVLALWNTDTPQDRVRH | |
| NO: 46 | | VYLSEAVRLRPDLESAQ | |
| T33-21A | trimer | RITTKVGDKGSTRLFGGEEVWKDSPIIEANGTLDELTSFIGEAKHYVDEEMK | |
| SEQ ID | | GILEEIQNDIYKIMGEIGSKGKIEGISEERIAWLLKLILRYMEMVNLKSFVL | |
| NO: 47 | | PGGTLESAKLDVCRTIARRALRKVLTVTREFGIGAEAAAYLLALSDLLFLLA | |
| | | RVIEIEKNKLKEVRS | |
| T33-21B | trimer | PHLVIEATANLRLETSPGELLEQANKALFASGQFGEADIKSRFVTLEAYRQG | |
| SEQ ID | | TAAVERAYLHACLSILDGRDIATRTLLGASLCAVLAEAVAGGGEEGVQVSVE | |
| NO: 48 | | VREMERLSYAKRVVARQR | |
| T33-28A | trimer | ESVNTSFLSPSLVTIRDFDNGQFAVLRIGRTGFPADKGDIDLCLDKMIGVRA | |
| SEQ ID | | AQIFLGDDTEDGFKGPHIRIRCVDIDDKHTYNAMVYVDLIVGTGASEVERET | |
| NO: 49 | | AEEEAKLALRVALQVDIADEHSCVTQFEMKLREELLSSDSFHPDKDEYYKDF | |
| | | L | |
| T33-28B | trimer | PVIQTFVSTPLDHHKRLLLAIIYRIVTRVVLGKPEDLVMMTFHDSTPMHFFG | |
| SEQ ID | | STDPVACVRVEALGGYGPSEPEKVTSIVTAAITAVCGIVADRIFVLYFSPLH | |
| NO: 50 | | CGWNGTNF | |
| T33-31A | trimer | EEVVLITVPSALVAVKIAHALVEERLAACVNIVPGLTSIYREEGSVVSDHEL | |
| SEQ ID | | LLLVKTTTDAFPKLKERVKELHPYEVPEIVALPIAEGNREYLDWLRENTG | |
| NO: 51 | | | |

TABLE 2

| | | | isoelectric | percent |
|---|---|---|---|---|
| name | MW | Oligomer MW | point | hydrophobic(“ILVMFW”) |
| I53-34A | 21427 | 64281 | 5.82 | 0.33 |
| I53-34B | 17083 | 85414 | 6.1 | 0.31 |
| I53-40A | 17091 | 85455 | 6.85 | 0.32 |
| I53-40B | 21789 | 65367 | 4.91 | 0.34 |
| I53-47A | 12191 | 36572 | 6.47 | 0.34 |
| I53-47B | 16956 | 84781 | 6.31 | 0.31 |
| I53-50A | 21783 | 65350 | 6.91 | 0.35 |
| I53-50B | 16839 | 84195 | 5.95 | 0.32 |
| I53-51A | 19967 | 59900 | 8.74 | 0.34 |
| I53-51B | 17178 | 85892 | 6.31 | 0.3 |
| I52-03A | 21026 | 105129 | 6.16 | 0.31 |
| I52-03B | 25875 | 51749 | 5.32 | 0.3 |
| I52-32A | 15015 | 30029 | 5.43 | 0.42 |
| I52-32B | 16877 | 84383 | 6.58 | 0.35 |
| I52-33A | 17914 | 89569 | 7.17 | 0.36 |
| I52-33B | 19215 | 38430 | 7.12 | 0.37 |
| I32-06A | 21632 | 43263 | 6.78 | 0.3 |
| I32-06B | 14736 | 44208 | 6.48 | 0.34 |
| I32-19A | 25405 | 76214 | 7.86 | 0.3 |
| I32-19B | 17186 | 34373 | 6.57 | 0.24 |
| I32-28A | 16648 | 49944 | 5.23 | 0.32 |
| I32-28B | 16173 | 32347 | 4.76 | 0.31 |
| I53-40A.1 | 17148 | 85740 | 7.66 | 0.32 |
| I53-40B.1 | 22070 | 66211 | 4.74 | 0.34 |
| I53-47A.1 | 12233 | 36698 | 5.65 | 0.34 |
| I53-47A.1NegT2 | 12209 | 36626 | 4.4 | 0.34 |
| I53-47B.1 | 17045 | 85226 | 6.04 | 0.31 |
| I53-47B.1NegT2 | 16902 | 84509 | 4.75 | 0.31 |
| I53-50A.1 | 21765 | 65296 | 6.17 | 0.35 |
| I53-50A.1NegT2 | 21746 | 65237 | 4.62 | 0.35 |
| I53-50A.1PosT1 | 21790 | 65369 | 9.07 | 0.35 |
| I53-50B.1 | 16926 | 84631 | 6.04 | 0.32 |
| I53-50B.1NegT2 | 16810 | 84049 | 4.8 | 0.32 |
| I53-50B.4PosT1 | 16867 | 50602 | 6.77 | 0.32 |
| I53-40A genus | 17091 | 85455 | 6.85 | 0.32 |
| I53-40B genus | 21789 | 65367 | 4.91 | 0.34 |
| I53-47A genus | 12191 | 36572 | 6.47 | 0.34 |
| I53-47B genus | 16956 | 84781 | 6.31 | 0.31 |
| I53-50A genus | 21652 | 64956 | 7.15 | 0.35 |
| I53-50B genus | 16839 | 84195 | 5.95 | 0.32 |
| T32-28A | 17168 | 34337 | 4.79 | 0.29 |
| T32-28B | 18711 | 56132 | 5.52 | 0.36 |
| T33-09A | 11321 | 33963 | 6.08 | 0.36 |
| T33-09B | 13279 | 39838 | 5.14 | 0.34 |
| T33-15A | 18902 | 56705 | 4.53 | 0.3 |
| T33-15B | 13298 | 39895 | 5.65 | 0.34 |
| T33-21A | 19158 | 57474 | 6.04 | 0.35 |
| T33-21B | 13128 | 39383 | 5.73 | 0.28 |
| T33-28A | 17637 | 52910 | 4.5 | 0.31 |
| T33-28B | 12302 | 36907 | 6.59 | 0.38 |
| T33-31A | 11329 | 33987 | 4.88 | 0.35 |
| T33_dn2A | 13632 | 40896 | 4.7 | 0.19 |
| T33_dn2B | 13687 | 41061 | 5.57 | 0.19 |
| T33_dn5A | 13528 | 40583 | 4.07 | 0.19 |
| T33_dn5A | 19741 | 59222 | 5.45 | 0.29 |
| T33_dn10A | 13883 | 41649 | 4.26 | 0.2 |
| T33_dn10B | 30222 | 90666 | 6.31 | 0.3 |
| 153_dn5A | 17004 | 85019 | 7.14 | 0.35 |
| 153_dn5B | 14138 | 70688 | 4.94 | 0.19 |

Tables 1 and 2 provides the amino acid sequence of the compA and compBs of embodiments of the present disclosure. In each case, the pairs of sequences together form an I53 multimer with icosahedral symmetry. The right-hand column in Table 1 identifies the residue numbers in each exemplary polypeptide that were identified as present at the interface of resulting assembled virus-like particles (i.e.: “identified interface residues”). As can be seen, the number of interface residues for the exemplary polypeptides of SEQ ID NO:1-34 range from 4-13. In some embodiments, compA and compB have 4, 5, 6, 7, 8, 9, 10, 11, 12, or 13 interface residues. In various embodiments, the compA and compB proteins comprise an amino acid sequence that is at least 75%, 80%, 85%, 90%, 91%, 92%, 93%, 94%, 95%, 96%, 97%, 98%, or 99% identical over its length, and identical to at least 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOS: 1-34. SEQ ID NOs: 35-51 represent other amino acid sequences of the compA and compBs from embodiments of the present disclosure. In other embodiments, the compA and/or compBs comprise an amino acid sequence that is at least 75%, 80%, 85%, 90%, 91%, 92%, 93%, 94%, 95%, 96%, 97%, 98%, or 99% identical over its length, and identical at least at 20%, 25%, 33%, 40%, 50%, 60%, 70%, 75%, 80%, 90%, or 100% of the identified interface positions, to the amino acid sequence of a polypeptide selected from the group consisting of SEQ ID NOS:1-51.

As shown in Table 2, the compA and compB proteins have similar molecular weights (MW), isoelectric points (pI) and percent hydrophobic residues, suggesting they can be expressed and purified using similar methods.

As is the case with proteins in general, the polypeptides are expected to tolerate some variation in the designed sequences without disrupting subsequent assembly into virus-like particles: particularly when such variation comprises conservative amino acid substitutions. As used here, “conservative amino acid substitution” means that: hydrophobic amino acids (Ala, Cys, Gly, Pro, Met, Val, Ile, Leu) can only be substituted with other hydrophobic amino acids; hydrophobic amino acids with bulky side chains (Phe, Tyr, Trp) can only be substituted with other hydrophobic amino acids with bulky side chains; amino acids with positively charged side chains (Arg, His, Lys) can only be substituted with other amino acids with positively charged side chains; amino acids with negatively charged side chains (Asp, Glu) can only be substituted with other amino acids with negatively charged side chains; and amino acids with polar uncharged side chains (Ser, Thr, Asn, Gln) can only be substituted with other amino acids with polar uncharged side chains.

In various embodiments of the pbVLPs of the invention, the compA and compBs, or the vice versa, comprise polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 95%, at least 99%, at least 100% over its length, and/or identical at least at one identified interface position, to the amino acid sequence indicated by the SEQ ID NO):
SEQ ID NO:1 and SEQ ID NO:2 (I53-34A and I53-34B);
SEQ ID NO:3 and SEQ ID NO:4 (I53-40A and I53-40B);
SEQ ID NO:3 and SEQ ID NO:24 (I53-40A and I53-40B.1);
SEQ ID NO:23 and SEQ ID NO:4 (I53-40A.1 and I53-40B);
SEQ ID NO:35 and SEQ ID NO:36 (I53-40A genus and I53-40B genus);
SEQ ID NO:5 and SEQ ID NO:6 (I53-47A and I53-47B);
SEQ ID NO:5 and SEQ ID NO:27 (I53-47A and I53-47B.1);
SEQ ID NO:5 and SEQ ID NO:28 (I53-47A and I53-47B.1NegT2);
SEQ ID NO:25 and SEQ ID NO:6 (I53-47A.1 and I53-47B);
SEQ ID NO:25 and SEQ ID NO:27 (I53-47A.1 and 153-47B.1);
SEQ ID NO:25 and SEQ ID NO:28 (I53-47A.1 and I53-47B.1NegT2);
SEQ ID NO:26 and SEQ ID NO:6 (I53-47A.1NegT2 and I53-47B);
SEQ ID NO:26 and SEQ ID NO:27 (I53-47A.1NegT2 and I53-47B.1);
SEQ ID NO:26 and SEQ ID NO:28 (I53-47A.1NegT2 and I53-47B.1NegT2);
SEQ ID NO:37 and SEQ ID NO:38 (I53-47A genus and I53-47B genus);
SEQ ID NO:7 and SEQ ID NO:8 (I53-50A and 153-50B);
SEQ ID NO:7 and SEQ ID NO:32 (I53-50A and I53-50B.1);
SEQ ID NO:7 and SEQ ID NO:33 (I53-50A and I53-50B.1NegT2);
SEQ ID NO:7 and SEQ ID NO:34 (I53-50A and I53-50B.4PosT1);
SEQ ID NO:29 and SEQ ID NO:8 (I53-50A.1 and 153-50B);
SEQ ID NO:29 and SEQ ID NO:32 (I53-50A.1 and 153-50B.1);
SEQ ID NO:29 and SEQ ID NO:33 (I53-50A.1 and I53-50B.1NegT2);
SEQ ID NO:29 and SEQ ID NO:34 (I53-50A.1 and I53-50B.4PosT1);
SEQ ID NO:30 and SEQ ID NO:8 (I53-50A.1NegT2 and 153-50B);
SEQ ID NO:30 and SEQ ID NO:32 (I53-50A.1NegT2 and 153-50B.1);
SEQ ID NO:30 and SEQ ID NO:33 (I53-50A.1NegT2 and I53-50B.1NegT2);
SEQ ID NO:30 and SEQ ID NO:34 (I53-50A.1NegT2 and I53-50B.4PosT1);
SEQ ID NO:31 and SEQ ID NO:8 (I53-50A.1PosT1 and 153-50B);
SEQ ID NO:31 and SEQ ID NO:32 (I53-50A.1PosT1 and 153-50B.1);
SEQ ID NO:31 and SEQ ID NO:33 (I53-50A.1PosT1 and I53-50B.1NegT2);
SEQ ID NO:31 and SEQ ID NO:34 (I53-50A.1PosT1 and I53-50B.4PosT1);
SEQ ID NO:39 and SEQ ID NO:40 (I53-50A genus and I53-50B genus);
SEQ ID NO:9 and SEQ ID NO:10 (I53-51A and I53-51B);
SEQ ID NO:11 and SEQ ID NO:12 (I52-03A and I52-03B);
SEQ ID NO:13 and SEQ ID NO:14 (I52-32A and I52-32B);
SEQ ID NO:15 and SEQ ID NO:16 (I52-33A and I52-33B)
SEQ ID NO:17 and SEQ ID NO:18 (I32-06A and I32-06B);
SEQ ID NO:19 and SEQ ID NO:20 (I32-19A and I32-19B);
SEQ ID NO:21 and SEQ ID NO:22 (I32-28A and I32-28B);
SEQ ID NO:23 and SEQ ID NO:24 (I53-40A.1 and I53-40B.1);
SEQ ID NO:41 and SEQ ID NO:42 (T32-28A and T32-28B);
SEQ ID NO:43 and SEQ ID NO:44 (T33-09A and T33-09B);
SEQ ID NO:45 and SEQ ID NO:46 (T33-15A and T33-15B);
SEQ ID NO:47 and SEQ ID NO:48 (T33-21A and T33-21B);
SEQ ID NO:49 and SEQ ID NO:50 (T33-28A and T32-28B); and
SEQ ID NO:51 and SEQ ID NO:44 (T33-31A and T33-09B (also referred to as T33-31B)), wherein those ending in “A” are compA and those ending in “B” are compB (e.g. 153-34A is compA and I53-34B is compB).

Non-limiting examples of designed protein complexes useful in protein-based VLPs of the present disclosure include those disclosed in U.S. Pat. No. 9,630,994; Int'l Pat. Pub No. WO2018187325A1; U.S. Pat. Pub. No. 2018/0137234 A1; U.S. Pat. Pub. No. 2019/0155988 A2, each of which is incorporated herein in its entirety.

In some embodiments, the disclosure provides pbVLPs comprising (i) a fusion protein of compA linked to an antigenic protein or antigenic fragment thereof and (ii) compB prepared according to a method described herein. The term “antigenic fragment” refers to any fragment of a protein that generates an immune response (humoral or T cell response) to the protein in vivo. The antigenic fragment may be a linear epitope, discontinuous epitope, or a conformation epitope (e.g., a folded domain). The antigenic fragment may preserve the secondary, tertiary, and/or quaternary structure of the full-length protein. In some embodiments, the antigenic fragment comprises a neutralizing epitope. In such cases, the VLP may generate a neutralizing antibody response. Antigenic fragments may be designed computationally, such as by predicting the secondary structure and rationally removing N- or C-terminal unstructured regions or internally loops, or entire structural elements (alpha helices and/or beta sheets).

In some embodiments, the antigenic protein is selected from HIV Env, RSV F, EBV gp350, CMV gB, CMV UL128, CMV UL130, CMV UL131A, CMV gH, CMV gL, Lyme OspA, Pertussis toxin, Dengue E, SARS S, MERS S, Zaire ebolavirus GP, Sudan ebolavirus GP, Marburg virus GP, Hanta virus Gn, Hanta virus Gc, HepB surface antigen, Measles H, Zika envelope domain III, Malaria CSP, Malaria Pfs25, Nipah virus F, Nipah virus G, Rotavirus VP4, Rotavirus VP8*, hMPV F, hMPV G, PV F, PV HN, MenB fHbp, MenB NadA, coronavirus S protein, coronavirus RBD, and MenB NHBA. In some embodiments, the antigenic protein is a paramyxovirus and/or pneumovirus F protein, or antigenic fragment thereof. Exemplary paramyxovirus and/or pneumovirus include, but are not limited to, respiratory syncytial virus (RSV) and Human metapneumovirus (hMPV). (C. L. Afonso et al., Taxonomy of the order Mononegavirales: update 2016. Arch. Virol. 161, 2351-2360 (2016)).

In some embodiments, the antigenic protein comprises an amino acid sequence having at least 75%, 80%, 85%, 90%, 91%, 92%, 93%, 94%, 95%, 96%, 97%, 98%, 99%, or 100% identity to an amino acid sequence selected from SEQ ID NOs: 64-136 and 206-209. In some embodiments, the antigenic protein comprises a signal peptide that is cleaved during processing. In some embodiments, the signal peptide comprises an amino acids sequence set forth by SEQ ID NOs: 210-212.

In some embodiments, the fusion protein comprises a linker positioned between the compA protein and the antigenic protein. In some embodiment, the fusion protein comprises an amino acid linker positioned between the compA protein and the antigenic protein. In some embodiments, the amino acid linker is a Gly-Ser linker (i.e.: a linker consisting of glycine and serine residues) of any suitable length. In some embodiments, the Gly-Ser linker is 3, 4, 5, 6, 7, 8, 9, 10, 11, 12, 13, 14, 15, 16, 17, 18, 19, 20, or more amino acids in length. In some embodiments, the Gly-Ser linker comprises or consists of an amino acid sequence selected from SEQ ID NOs: 213-215.

In some embodiments, the fusion protein comprises an amino acid sequence having at least 75%, 80%, 85%, 90%, 91%, 92%, 93%, 94%, 95%, 96%, 97%, 98%, 99%, or 100% identity to an amino acid sequence selected from SEQ ID NOs: 61 and 167-193. In some embodiments, the fusion protein comprises an amino acid sequence having at least 75%, 80%, 85%, 90%, 91%, 92%, 93%, 94%, 95%, 96%, 97%, 98%, 99%, or 100% identity to an amino acid sequence selected from SEQ ID NOs: 137-166 and 194-199. In some embodiments, the fusion protein comprises an amino acid sequence having at least 75%, 80%, 85%, 90%, 91%, 92%, 93%, 94%, 95%, 96%, 97%, 98%, 99%, or 100% identity to an amino acid sequence selected from SEQ ID NOs: 200-205.

Table 3 provides illustrative sequences for fusion proteins comprising a CompA component linked to an antigenic protein derived from human metapneumovirus (hMPV) or respiratory syncytial virus (RSV).

TABLE 3
Amino acid sequences for exemplary VLP components

| Name/ | | SEQ |
|---|---|---|
| Identifier | Sequence | ID NO |
| CompA- | KESYLEESCSTITEGYLSVLRTGWYTNVFTLEVGDVENLTCSD | 60 |
| hMPV-his | GPSLIKTELDLTKSALRELKTVSADQLAREEQIENPRQSRFVLG | |
| | AIALGVATAAAVTAGVAIAKTIRLESEVTAIKNALKTTNEAVS | |
| | TLGNGVRVLATAVRELKDFVSKNLTRAINKNKCDIDDLKMA | |
| | VSFSQFNRRFLNVVRQFSDNAGITPAISLDLMTDAELARAVSN | |
| | MPTSAGQIKLMLENRAMVRRKGFGILIGVYGSSVIYMVQLPIF | |
| | GVIDTPCWIVKAAPSCSEKKGNYACLLREDQGWYCQNAGST | |
| | VYYPNEKDCETRGDHVFCDTAAGINVAEQSKECNINISTTNYP | |
| | CKVSTGRHPISMVALSPLGALVACYKGVSCSIGSNRVGIIKQL | |
| | NKGCSYITNQDADTVTIDNTVYQLSKVEGEQHVIKGRPVSSSF | |
| | DPIKFPEDQFNVALDQVFENIENSQAGSGGSGSGSGGSEKAAK | |
| | AEEAARKMEELFKKHKIVAVLRANSVEEAIEKAVAVFAGGV | |
| | HLIEITFTVPDADTVIKALSVLKEKGAIIGAGTVTSVEQARKAV | |
| | ESGAEFIVSPHLDEEISQFAKEKGVFYMPGVMTPTELVKAMK | |
| | LGHTILKLFPGEVVGPQFVKAMKGPFPNVKFVPTGGVNLDNV | |
| | AEWFKAGVLAVGVGSALVKGTPDEVREKAKAFVEKIRGATE | |
| | LE | |
| CompA- | MELLILKANAITTILTAVTFCFASGQNITEEFYQSTCSAVSKGY | 61 |
| RSV-02 | LSALRTGWYTSVITIELSNIKENKCNGTDAKVKLIKQELDKYK | |
| | NAVTELQLLMQSTPATNNRARRELPRFMNYTLNNAKKTNVT | |
| | LSKKRKRRFLGFLLGVGSAIASGVAVCKVLHLEGEVNKIKSA | |
| | LLSTNKAVVSLSNGVSVLTFKVLDLKNYIDKQLLPILNKQSCS | |
| | ISNIETVIEFQQKNNRLLEITREFSVNAGVTTPVSTYMLTNSEL | |
| | LSLINDMPITNDQKKLMSNNVQIVRQQSYSIMCIIKEEVLAYV | |
| | VQLPLYGVIDTPCWKLHTSPLCTTNTKEGSNICLTRTDRGWY | |
| | CDNAGSVSFFPQAETCKVQSNRVFCDTMNSLTLPSEVNLCNV | |
| | DIFNPKYDCKIMTSKTDVSSSVITSLGAIVSCYGKTKCTASNK | |
| | NRGIIKTFSNGCDYVSNKGVDTVSVGNTLYYVNKQEGKSLYV | |
| | KGEPIINFYDPLVFPSDEFDASISQVNEKINQSLAFIRKSDELLG | |
| | SGGSGSGSGGSEKAAKAEEAARKMEELFKKHKIVAVLRANS | |
| | VEEAIEKAVAVFAGGVHLIEITFTVPDADTVIKALSVLKEKGAI | |
| | IGAGTVTSVEQCRKAVESGAEFIVSPHLDEEISQFCKEKGVFY | |
| | MPGVMTPTELVKAMKLGHTILKLFPGEVVGPQFVKAMKGPF | |
| | PNVKFVPTGGVNLDNVCEWFKAGVLAVGVGSALVKGTPDE | |
| | VREKAKAFVEKIRGCTE | |
| CompB-01 | MNQHSHKDHETVRIAVVRARWHAEIVDACVSAFEAAMRDIG | 63 |
| | GDRFAVDVFDVPGAYEIPLHARTLAETGRYGAVLGTAFVVN | |
| | GGIYRHEFVASAVINGMMNVQLNTGVPVLSAVLTPHNYDKS | |
| | KAHTLLFLALFAVKGMEAARACVEILAAREKIAA | |

In various embodiments of the pbVLPs of the disclosure, the compA and compB proteins comprise polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 95%, at least 99%, at least 100% over its length, and/or identical at least at one identified interface position, to the amino acid sequence indicated by the SEQ ID NO):
SEQ ID NO:52 and SEQ ID NO:53 (T33_dn2A and T33_dn2B);
SEQ ID NO:54 and SEQ ID NO:55 (T33_dn5A and T33_dn5B);
SEQ ID NO:56 and SEQ ID NO:57 (T33_dn10A and T33_dn10B); or
SEQ ID NO:58 and SEQ ID NO:59 (153 dn5A and 153 dn5B),
   wherein those ending in “dn5B” are compA and those ending in “dn5A” are compB (e.g. I53_dn5B is compA and dn5A is compB).

T33_dn2A
(SEQ ID NO: 52)
NLAEKMYKAGNAMYRKGQYTIAIIAYTLALLKDPNNAEAWYNLGNAAYKK
GEYDEAIEAYQKALELDPNNAEAWYNLGNAYYKQGDYDEAIEYYKKALRL
DPRNVDAIENLIEAEEKQG
T33_dn2B
(SEQ ID NO: 53)
EEAELAYLLGELAYKLGEYRIAIRAYRIALKRDPNNAEAWYNLGNAYYKQ
GDYREAIRYYLRALKLDPENAEAWYNLGNALYKQGKYDLAIIAYQAALEE
DPNNAEAKQNLGNAKQKQG
T33_dn5A
(SEQ ID NO: 54)
NSAEAMYKMGNAAYKQGDYILAIIAYLLALEKDPNNAEAWYNLGNAAYKQ
GDYDEAIEYYQKALELDPNNAEAWYNLGNAYYKQGDYDEAIEYYEKALEL
DPNNAEALKNLLEAIAEQD
T33_dn5B
(SEQ ID NO: 55)
TDPLAVILYIAILKAEKSIARAKAAEALGKIGDERAVEPLIKALKDEDAL
VRAAAADALGQIGDERAVEPLIKALKDEEGLVRASAAIALGQIGDERAVQ
PLIKALTDERDLVRVAAAVALGRIGDEKAVRPLIIVLKDEEGEVREAAAI
ALGSIGGERVRAAMEKLAERGTGFARKVAVNYLETHK
T33_dn10A
(SEQ ID NO: 56)
EEAELAYLLGELAYKLGEYRIAIRAYRIALKRDPNNAEAWYNLGNAYYKQ
GDYDEAIEYYQKALELDPNNAEAWYNLGNAYYKQGDYDEAIEYYEKALEL
DPENLEALQNLLNAMDKQG
T33_dn10B
(SEQ ID NO: 57)
IEEVVAEMIDILAESSKKSIEELARAADNKTTEKAVAEAIEEIARLATAA
IQLIEALAKNLASEEFMARAISAIAELAKKAIEAIYRLADNHTTDTFMAR
AIAAIANLAVTAILAIAALASNHTTEEFMARAISAIAELAKKAIEAIYRL
ADNHTTDKFMAAAIEAIALLATLAILAIALLASNHTTEKFMARAIMAIAI
LAAKAIEAIYRLADNHTSPTYIEKAIEAIEKIARKAIKAIEMLAKNITTE
EYKEKAKKIIDIIRKLAKMAIKKLEDNRT
I53_dn5A
(SEQ ID NO: 58)
KYDGSKLRIGILHARWNAEIILALVLGALKRLQEFGVKRENIIIETVPGS
FELPYGSKLFVEKQKRLGKPLDAIIPIGVLIKGSTMHFEYICDSTTHQLM
KLNFELGIPVIFGVLTCLTDEQAEARAGLIEGKMHNHGEDWGAAAVEMAT
KFN
I53_dn5B
(SEQ ID NO: 59)
EEAELAYLLGELAYKLGEYRIAIRAYRIALKRDPNNAEAWYNLGNAYYKQ
GRYREAIEYYQKALELDPNNAEAWYNLGNAYYERGEYEEAIEYYRKALRL
DPNNADAMQNLLNAKMREE

Assembly of pbVLPs

In some embodiments, a single component self-assembles into the pbVLP. In some embodiments, the single component is compA. In some embodiments, one or more purified samples of first and second components for use in forming a pbVLP are mixed in an approximately equimolar molar ratio in aqueous conditions (e.g., an I53-50A/B icosahedral pbVLP). In some embodiments, the first and second components are compA and compB respectively. The first and second components (through the multimerization domains and optionally through the ectodomains) interact with one another to drive assembly of the target pbVLP. Successful assembly of the target pbVLP can be confirmed by analyzing the in vitro assembly reaction by common biochemical or biophysical methods used to assess the physical size of proteins or protein assemblies, including but not limited to size exclusion chromatography, native (non-denaturing) gel electrophoresis, dynamic light scattering, multi-angle light scattering, analytical ultracentrifugation, negative stain electron microscopy, cryo-electron microscopy, or X-ray crystallography. If necessary, the assembled pbVLP can be purified from other species or molecules present in the in vitro assembly reaction using preparative techniques commonly used to isolate proteins by their physical size, including but not limited to size exclusion chromatography, preparative ultracentrifugation, tangential flow filtration, or preparative gel electrophoresis. The presence of the antigenic protein in the pbVLP can be assessed by techniques commonly used to determine the identity of protein molecules in aqueous solutions, including but not limited to SDS-PAGE, mass spectrometry, protein sequencing, ELISA, surface plasmon resonance, biolayer interferometry, or amino acid analysis. The accessibility of the protein on the exterior of the particle, as well as its conformation or antigenicity, can be assessed by techniques commonly used to detect the presence and conformation of an antigen, including but not limited to binding by monoclonal antibodies, conformation-specific monoclonal antibodies, surface plasmon resonance, biolayer interferometry, or antisera specific to the antigen.

In various embodiments, the pbVLPs of the disclosure comprise two or more distinct compAs bearing different antigenic proteins as genetic fusions; these pbVLPs co-display multiple different proteins on the same pbVLP. These multi-antigen pbVLPs are produced by performing in vitro assembly with mixtures of two or more antigens each comprising a multimerization domain. The fraction of each antigen in the mixture determines the average valency of each antigenic protein in the resulting pbVLPs. The presence and average valency of each antigen in a given sample can be assessed by quantitative analysis using the techniques described above for evaluating the presence of antigenic proteins in full-valency pbVLPs.

In various embodiments, the pbVLPs are between about 20 nanometers (nm) to about 40 nm in diameter, with interior lumens between about 15 nm to about 32 nm across and pore sizes in the protein shells between about 1 nm to about 14 nm in their longest dimensions.

In some embodiments, the pbVLPs has icosahedral symmetry. In such embodiment, the pbVLP may comprise 60 copies of a first component and 60 copies of a second component. In one such embodiment, the number of identical compAs in each first assembly is different than the number of identical compAs in each second assembly. For example, in some embodiments, the pbVLP comprises twelve first assemblies and twenty second assemblies; in such embodiments, each first assembly may, for example, comprise five copies of the identical first component, and each second assembly may, for example, comprise three copies of the identical second component. In other embodiments, the pbVLP comprises twelve first assemblies and thirty second assemblies; in such an embodiment, each first assembly may, for example, comprise five copies of the identical first component, and each second assembly may, for example, comprise two copies of the identical second component. In further embodiments, the pbVLP comprises twenty first assemblies and thirty second assemblies; in this embodiment, each first assembly may, for example, comprise three copies of the identical first component, and each second assembly may, for example, comprise two copies of the identical second component. All of these embodiments are capable of forming protein-based VLPs with regular icosahedral symmetry.

In various further embodiments, oligomeric states of the first and second multimerization domains are as follows:
I53-34A: trimer+I53-34B: pentamer;
I53-40A: pentamer+I53-40B: trimer;
I53-47A: trimer+I53-47B: pentamer;
I53-50A: trimer+I53-50B: pentamer;
I53-51A: trimer+I53-51B: pentamer;
I32-06A: dimer+I32-06B: trimer;
I32-19A: trimer+I32-19B: dimer;
I32-28A: trimer+I32-28B: dimer;
I52-03A: pentamer+I52-03B: dimer;
I52-32A: dimer+I52-32B: pentamer; and
I52-33A: pentamer+I52-33B: dimer

### Methods of Making Nanostructures

The present disclosure provides methods of combining one or more proteins (e.g., compA and/or compB) under conditions that yield formation of protein nanostructures. In some embodiments, the method comprises addition of a first protein (e.g., compA) to a solution comprising a second protein (e.g., compB), wherein the addition provides a partially or substantially homogenous solution of the first and second proteins, i.e., having consistent or uniform physical composition throughout the solution, and wherein the first and second proteins assemble to form nanostructures. Mechanical mixing, e.g., with a mixing blade, stir bar, or impeller, is often used to mix miscible liquids. However, as described herein, it was found the addition of compA to a solution of compB using mechanical mixing resulted in a composition of pbVLPs having substantial turbidity due to precipitation of compA and/or compB. In contrast, it was discovered that the addition of compA to a solution of compB under conditions that induced blending of compA and compB with minimized shear stress (e.g., addition of compA under the surface of the compB solution and/or additionally without mechanical stirring) resulted in formation of pbVLPs with minimal or limited precipitation of compA and/or compB. Moreover, it was demonstrated that robust, homogenous, and rapid liquid-to-liquid mixing of a solution containing compA and a solution containing compB is achieved using microfluidics mixing, thereby enabling formation of a nanostructure (e.g., pbVLP) comprising compA and compB.

Accordingly, the present disclosure provides methods for (i) addition of a first protein (e.g., compA) to a solution comprising a second protein (e.g., compB), and (ii) mixing the first protein and second protein under conditions that minimizes shear stress, thereby resulting in formation of a protein nanostructure (e.g., pbVLP). In some embodiments, the method of mixing is any method known in the art suitable for blending, mixing, or combining miscible liquids (e.g., low viscosity liquids) under conditions that minimize shear stress, e.g., as compared to mechanical mixing using a blade, stir bar, or impeller. As described herein, “shear stress” refers to the differences in velocity throughout a fluid, e.g., in a flow vessel subjected to agitation, wherein the greater the velocity differences within the fluid, the greater the degree of shear stress. Methods for measuring shear stress in a fluid are known in the art, and include, e.g., laser doppler velocimetry.

In some embodiments, the method of mixing, blending, or combining the first protein (e.g., compA) and the second protein (e.g., compB) under conditions that minimize shear stress provides a suspension of protein nanostructures (e.g., pbVLPs) having reduced turbidity as compared to mixing of the first and second protein by mechanical mixing e.g., using a blade, stir bar, or impeller. Methods of measuring turbidity using optical measurements or optical scattering measurements are known in the art. In some embodiments, the conditions that minimize shear stress result in formation of protein nanostructures (e.g., pbVLPs) with reduced precipitation of the nanostructure, the first protein (e.g., compA), and/or the second protein (e.g., compB) as compared to mechanical mixing e.g., using a blade, stir bar, or impeller.

In some embodiments, the conditions that minimize shear stress provide for reduced shear stress as compared to mechanical mixing following the addition of the first protein (e.g., compA) to a solution comprising the second protein (e.g., compB). In some embodiments, the conditions that minimize shear stress comprise mixing the solution without mechanical mixing. In some embodiments, the conditions that minimize shear stress comprise mixing the solution without a stir bar. In some embodiments, the conditions that minimize shear stress comprise mixing without an impeller.

In some embodiments, the method of mixing, blending, or combining the first protein (e.g., compA) and the second protein (e.g., compB) under conditions that minimize shear stress comprise orbital agitation. In some embodiments, the solution comprising the first and second protein is contained in a vessel that undergoes motion by orbital agitation using an orbital shaking platform. In some embodiments, the motion induced by orbital shaking provides for sufficient bulk mixing of the first protein (e.g., compA) and the second protein (e.g., compB) to form a partially or substantially homogenous mixture of the first protein and the second protein, thereby enabling formation of nanostructures comprising the first protein and the second protein (e.g., pbVLPs). In some embodiments, the containment vessel comprises a working volume exceeding about 100 mL (e.g., 250 mL, 500 mL, 750 mL). In some embodiments, the containment vessel comprises a working volume exceeding about 1 L, 10 L, 50 L, 100 L, 250 L, 500 L, 750 L, or 1000 L. In some embodiments, the orbital agitation is performed at ambient temperature (e.g., about 20-25° C.). In some embodiments, the orbital agitation is performed at an elevated temperature (e.g., about 30-37° C.). In some embodiments, the speed of the orbital agitation is selected to minimize shear stress of the mixing. In some embodiments, the containment vessel is agitated at a speed of about 30-100 rpm. In some embodiments, the containment vessel is agitated at a speed of about 70 rpm. In some embodiments, the containment vessel is agitated at a speed of about 80 rpm. In some embodiments, the containment vessel is agitated at a speed of about 90 rpm.

In some embodiments, the method of mixing, blending, or combining the first protein (e.g., compA) and the second protein (e.g., compB) under conditions that minimize shear stress comprise providing (i) a first inlet fluid stream, (ii) a second inlet fluid stream, and (iii) an outlet stream, wherein (i), (ii), and (iii) are joined at a branch point in a three-way configuration (e.g., a T-shaped configuration, a Y-shaped configuration), and wherein the fluid stream of (i) and the fluid stream of (ii) are contacted to form the outlet stream of (iii). In some embodiments, the first inlet fluid stream comprises a solution of the first protein (e.g., compA), the second inlet fluid stream comprises a solution of the second protein (e.g., compB). In some embodiments, a mixing, blending, or combining of the first protein and the second protein occurs in the outlet stream. In some embodiments, the mixing, blending, or combining results in formation of a nanostructure comprising the first protein and the second protein (e.g., pbVLP).

In some embodiments, the mixing, blending, or combining of the first protein (e.g., compA) and the second protein (e.g., compB) in the outlet stream is mediated by a static mixing element. A “static mixing element” or “static mixer” refers to a device inserted into a flow path for the purpose of facilitating mixing by causing the flow stream to divide, recombine, accelerate/decelerate, spread, swirl, or form layers without the use of moving parts. Static mixing devices are known in the art, and typically consist of individual mixing elements (e.g., plates, baffles, helical elements, or geometric grids) positioned at precise angles to direct flow and increase turbulence of the flow stream. Such devices are capable of mixing liquids having comparable or very different viscosities and volumetric flow rates. It is within the knowledge of the skilled person to select a static mixing device having particular mixing elements, cross section of a particular shape and size, and device length to facilitate uniform mixing of two or more inlet fluid streams. Methods to evaluate the quality of the mixing include measuring the radial variation coefficient (CoV), which describes the deviations of local concentration from the mean within a cross section of the flow path, and wherein a lower CoV indicates more uniform mixing. In some embodiments, the static mixing element is selected to achieve a CoV of less than about 0.05 (i.e., 95% of all concentration measurements to be taken from flow path cross section are within ±10%).

In some embodiments, the solution of the first protein (e.g., compA) is pumped through a first line, e.g., via a pump, to form the first inlet fluid stream. In some embodiments, the solution of the first protein (e.g., compA) is pumped through the first line at a flow rate of at least about 100 mL/min. In some embodiments, the solution of the first protein (e.g., compA) is pumped through the first line at a flow rate of about 100 mL/min, about 200 mL/min, about 300 mL/min, about 400 mL/min, about 500 mL/min, about 600 mL/min, about 700 mL/min, about 800 mL/min, or about 900 mL/min. In some embodiments, the solution of the first protein (e.g., compA) is pumped through the first line at a flow rate of about 1 L/min or greater than 1 L/min (e.g., about 2 L/min, about 3 L/min, about 4 L/min, about 5 L/min, about 6 L/min, about 7 L/min, about 8 L/min, about 9 L/min, about 10 L/min, about 20 L/min, about 30 L/min, about 40 L/min or about 50 L/min).

In some embodiments, solution of the second protein (e.g., compB) is pumped through a second line, e.g., via a pump, to form the second inlet fluid stream. In some embodiments, the solution of the second protein (e.g., compB) is pumped through the second line at a flow rate of at least about 100 mL/min. In some embodiments, the solution of the second protein (e.g., compB) is pumped through the second line at a flow rate of about 100 mL/min, about 200 mL/min, about 300 mL/min, about 400 mL/min, about 500 mL/min, about 600 mL/min, about 700 mL/min, about 800 mL/min, or about 900 mL/min. In some embodiments, the solution of the second protein (e.g., compB) is pumped through the second line at a flow rate of about 1 L/min or greater than 1 L/min (e.g., about 2 L/min, about 3 L/min, about 4 L/min, about 5 L/min, about 6 L/min, about 7 L/min, about 8 L/min, about 9 L/min, about 10 L/min, about 20 L/min, about 30 L/min, about 40 L/min or about 50 L/min).

In some embodiments, the first inlet stream comprising the solution of the first protein (e.g., compA) and the second inlet stream comprising the solution of the second protein (e.g., compB) are combined to form the outlet stream and passed through the static mixing element. In some embodiments, the first inlet stream and the second inlet stream are combined to form the outlet stream within the static mixing element. In some embodiments, the outlet stream comprises the solution of the first protein (e.g., compA) and the solution of the second protein (e.g., compB), wherein the solution of the first protein and the solution of the second protein are substantially homogenous, i.e., uniformly mixed, when the outlet stream exits the static mixing element.

Methods of Making Nanostructures using Microfluidics

In some embodiments, the method of mixing, blending, or combining the first protein (e.g., compA) and the second protein (e.g., compB) under conditions that minimize shear stress comprise microfluidic mixing. In some embodiments, the disclosure provides methods for making a nanostructure (e.g., pbVLP) comprising one or more protein components (e.g., compA and/or compB) using microfluidic mixing (see, e.g., Whitesides, George, M. Nature (2006) 442:368-373; Stroock, et al. Science (2002) 295:647-651; Valencia et al ACS Nano (2013) DOI/101.1021/nn403370e). As a non-limiting example, controlled microfluidic formulation includes a passive method for mixing streams of steady pressure-driven flows in micro channels at a low Reynolds number as described, e.g., in Stroock, et al. Science (2002) 295:647-651.

Microfluidics is a term commonly used to describe the study of fluid flow behavior inside channels of sub-millimeter cross-section. The microfluidic device is characterized in that physical phenomena generally classified under microtechnology are relevant in the fluidic channels and chambers arranged therein. These include, for example, capillary effects, and effects (especially mechanical effects) associated with surface tensions of the fluid. These additionally include effects such as thermophoresis and electrophoresis. In microfluidics, said phenomena are usually dominant over effects such as gravity. The microfluidic device can also be characterized in that it is produced at least in part using a layer-by-layer method and channels are arranged between layers of the layer structure. A microfluidic arrangement of chambers and interconnecting channels may be provided on a hydrophilic substrate, such as polydimethylsiloxane (PDMS), for convenient transport/manipulation of fluid or solutes from one chamber to another or within a channel. A substrate having a microfluidic arrangement like this may be called a microfluidic “chip” or “device.” The term “microfluidic” can also be characterized via the cross-sections or channels within the device that serve for guiding the fluid. For example, cross-sections are usually in the range from about 100 μm×100 μm to about 800 μm×800 μm. Exemplary microfluidic chips or microfluidic devices comprise a body of rigid material, e.g., thermoplastic material, comprising one or more fluid inlets, channels and mixing regions, and one or more outlets. Microfluidic devices are further described in U.S. Application Pub. Nos. 2020/0023358; 2012/0276209; 2014/0328759; 2016/0022580; and 2018/0111830, which are each incorporated herein by reference in their entirety.

In some embodiments, the method for making a nanostructure (e.g., pbVLP) comprising one or more protein components (e.g., compA and/or compB) comprises mixing of a first protein (e.g., compA) and a second protein (e.g., compB) using a microfluidic device. In some embodiments, the microfluidic device comprises (i) a first inlet for receiving a solution of the first protein (e.g., CompA); (ii) a second inlet for receiving a solution of the second protein (e.g., CompB); (iii) a first channel in fluid communication with the first inlet to provide a first inlet fluid stream comprising the solution of the first protein; (iv) a second channel in fluid communication with the second inlet to provide a second inlet fluid stream comprising the solution of the second protein; (v) an overlap region for receiving the first and second inlet fluid streams; and (vi) a third channel in fluid communication with the overlap region, wherein the first and second inlet fluid streams must flow through the third channel to form an outlet fluid stream, and wherein mixing of the solution of the first protein and the solution of the second protein occurs within the outlet fluid stream, thereby forming the nanostructures (e.g., VLPs) comprising the first protein and second protein.

As used herein, the term “channel” refers to a conduit of any desired shape or configuration through which a fluid stream is capable of being passed or directed. In some embodiments, the one or more of the dimensions of the channel are sub-millimeter (e.g., less than 500 microns). In some embodiments, the cross-section of the first channel, the second channel, and the third channel are each independently selected from: rectangular, round, square, circular, oval, ellipsoidal, or semi-circular. In some embodiments, the cross-section of the first channel has one or more dimensions less than 1 mm. In some embodiments, the cross-section of the first channel has one or more dimensions between about 30 nm and about 300 nm. In some embodiments, the cross-section of the second channel has one or more dimensions less than 1 mm. In some embodiments, the cross-section of the second channel has one or more dimensions between about 30 nm and about 300 nm. In some embodiments, the cross-section of the third channel has one or more dimensions less than 1 mm. In some embodiments, the cross-section of the third channel has one or more dimensions between about 30 nm and about 300 nm.

As used herein, the term “overlap region” refers to a zone of the microfluidic device wherein two or more channels comprising inlet fluid streams form a junction, wherein the inlet fluid streams have a principal flow direction towards the overlap region, and wherein the inlet fluid streams of the two or more channels are in fluid communication within the overlap region. In some embodiments, the microfluidic device comprises an overlap region between the first channel and the second channel. In some embodiments, the overlap region joins with the third channel. In some embodiments, the first channel, the second channel, and the third channel converge at the overlap region. In some embodiments, the overlap region receives the fluid stream in the first channel and the fluid stream in the second channel. In some embodiments, the fluid stream flows through the overlap region into the third channel. In some embodiments, the first channel, the second channel, and the third channel converge via a T-shaped or a Y-shaped overlap region.

In some embodiments, the third channel comprises a mixing channel. Micromixers for on-chip mixing have been developed based on different mechanisms to disturb laminar flow (see, e.g., Ward, K. (2015) 1 Micromech. Microeng. 25:094001). Such micromixers can be divided into two groups: passive and active micromixers (see, e.g., Nguyne and Wu (2005)J. Micromech. Microeng. 15:1). An active mixers rely on some form of external energy disturbance to generate a chaotic flow pattern in the microchannel. Typically, the external energy force is generated by a moving element in the micromixer itself, e.g., magnetically-actuated stirrers, or by the application of an external electrical force, e.g., pressure, ultrasound, acoustic, electrohydrodynamics, dielectrophoresis, electrokinetics, magnetohydrodynamics, thermal, etc. A passive mixing channel provides for mixing of two or more inlet fluid streams in the absence of turbulent flow conditions and without the use of moving elements. A passive mixing channel relies on the geometrical layout of the microchannels to cause lamination and/or chaotic advection. Designs for passive mixing channels include, but are not limited to, lamination-based designs that split streams and rejoin them after a certain distance (see, e.g., Ansari, et al. (2009) Chem Eng J. 146:439); staggered-herringbone design (see, e.g., Stroock, et al (2002) Science 295:647), and planar spiral microchannels (see, e.g., Sudarsan, et al (2006) Lab on a Chip, 6:74-82);

In some embodiments, the third channel comprises a passive mixing channel. In some embodiments, the passive mixing channel has a principal flow direction comprising laminar flow and comprises one or more passive elements to facilitate mixing. In some embodiments, the one or more passive elements comprises stream splitting and recombination. In some embodiments, the one or more passive elements comprises slanted wells, ridges, or grooves. In some embodiments, the one or more passive elements comprises channel overlaps, slits, converging/diverging regions, turns, and/or apertures, wherein the one or more passive elements promotes rapid and controlled mixing between two or more fluid streams. In some embodiments, a passive mixing channel suitable for use in the present disclosure is any known in the art, see e.g.; International Publication Number WO 97/00125; U.S. Pat. Nos. 6,890,093; 6,264,900; 7,160,025; and U.S. Application Nos. 2004/0262223; 2007/026377; 2021/0069699; 2020/0023358. In some embodiments, the third channel comprises a first region adapted for flowing the first and second inlet fluid streams under laminar flow conditions and a second region that is a passive mixing channel. In some embodiments, the second region comprises an active micromixer. In some embodiments, the third channel enables mixing of the contents of the first and second inlet fluid streams under conditions that provide nanostructures (e.g., VLPs) comprising the first protein and second protein.

In some embodiments, a method of the disclosure for making a nanostructure (e.g., pbVLPs) comprising one or more protein components (e.g., compA and/or compB) comprises: (i) introducing a first fluid stream comprising a solution of a first protein (e.g., compA) into the first inlet of the microfluidic device, (ii) introducing a second fluid stream comprising a solution of a second protein (e.g., compB) into the second inlet fluid stream of the microfluidic device, (iii) flowing the first inlet fluid stream under laminar flow conditions through the first channel of the microfluidic device into the third channel of the microfluidic device; and (iv) flowing the second inlet fluid stream under laminar flow conditions through the second channel of the microfluidic device into the third channel of the microfluidic device, wherein the first inlet fluid stream and the second inlet fluid stream form the outlet fluid stream, wherein mixing of the contents of the first fluid stream and the second fluid stream occurs in the third channel, thereby providing nanostructures (e.g., pbVLPs) comprising the one or more protein components.

In some embodiments, the solution of the first protein (e.g., compA) is pumped through the first channel using a first microfluidic pump, e.g., a syringe pump. In some embodiments, the solution of the first protein (e.g., compA) is pumped through the first channel at a flow rate of about 1 mL/min to about 20 mL/min. In some embodiments, the solution of the first protein (e.g., compA) is pumped through the first channel at a flow rate of about 2 mL/min. In some embodiments the solution of the first protein (e.g., compA) is pumped through the first channel at a flow rate of about 10 mL/min.

In some embodiments, the solution of the second protein (e.g., compB) is pumped through the second channel using a second microfluidic pump, e.g., a syringe pump. In some embodiments, the solution of the second protein (e.g., compB) is pumped through the second channel at a flow rate of about 1 mL/min to about 20 mL/min. In some embodiments, the solution of the second protein (e.g., compB) is pumped through the second channel at a flow rate of about 2 mL/min. In some embodiments the second solution is pumped through the second channel at a flow rate of about 10 mL/min. In some embodiments, the solution of the second protein (e.g., compB) is pumped through the second channel at a flow rate of about 10 mL/min, about 100 mL/min, about 200 mL/min, about 300 mL/min, about 400 mL/min, about 500 mL/min, about 600 mL/min, about 700 mL/min, about 800 mL/min, or about 900 mL/min. In some embodiments, the solution of the second protein (e.g., compB) is pumped through the second channel at a flow rate of about 1 L/min or greater than 1 L/min (e.g., about 2 L/min, about 3 L/min, about 4 L/min, about 5 L/min, about 6 L/min, about 7 L/min, about 8 L/min, about 9 L/min, about 10 L/min, about 20 L/min, about 30 L/min, about 40 L/min or about 50 L/min).

In some embodiments, the flow rate for the solution of the first protein (e.g., compA) being pumped through the first channel is equivalent to the flow rate being used to pump the solution of the second protein (e.g., compB) through the second channel. In some embodiments, the flow rate for the solution of the first protein (e.g., compA) being pumped through the first channel is greater than the flow rate being used to pump the solution of the second protein (e.g., compB) through the second channel. In some embodiments, the flow rate for the solution of the first protein (e.g., compA) being pumped through the first channel is less than the flow rate being used to pump the solution of the second protein (e.g., compB) through the second channel.

In some embodiments, a method of the disclosure for making a nanostructure (e.g., pbVLPs) comprising one or more protein components (e.g., compA and/or compB) comprises: (i) introducing a first fluid stream comprising a solution of a first protein (e.g., compA) into the first inlet of the microfluidic device at a first flow rate, wherein the first flow rate is about 2 ml/min to about 15 mL/min (ii) introducing a second fluid stream comprising a solution of a second protein (e.g., compB) into the second inlet fluid stream of the microfluidic device at a second flow rate, wherein the second flow rate is about 2 mL/min to about 15 mL/min (iii) flowing the first inlet fluid stream under laminar flow conditions through the first channel of the microfluidic device into the third channel of the microfluidic device; and (iv) flowing the second inlet fluid stream under laminar flow conditions through the second channel of the microfluidic device into the third channel of the microfluidic device, wherein mixing of the contents of the first fluid stream and the second fluid stream occurs in the third channel, and wherein the third channel provides an outlet fluid stream comprising nanostructures (e.g., pbVLPs) comprising the one or more protein components. In some embodiments, the rate of mixing is controlled by the first flow rate and the second flow rate. In some embodiments, the first flow rate and the second flow rate are selected to achieve rapid mixing. In some embodiments, the ratio of the first flow rate and the second flow rate is 1:1. In some embodiments, the first flow rate is greater than the second flow rate, wherein the ratio of the first flow rate and the second flow rate is 2:1, 3:1, 4:1, 5:1, 6:1, 7:1, 8:1, 9:1, or 10:1. In some embodiments, the second flow rate is greater than the first flow rate, wherein the ratio of the second flow rate and the first flow rate is 2:1, 3:1, 4:1, 5:1, 6:1, 7:1, 8:1, 9:1, or 10:1.

In some embodiments, the solution of the first protein (e.g., compA) comprises a molar concentration of the first protein (e.g., compA), and the solution of the second protein (e.g., compB) comprises a molar concentration of the second protein (e.g., compB), wherein the molar concentration of the first protein is substantially equivalent to the molar concentration of the second protein (i.e., about 1:1). In some embodiments, the molar concentration of the first protein (e.g., compA) is greater than the molar concentration of the second protein (e.g., compB). In some embodiments, the molar concentration of the first protein (e.g., compA) is greater than the molar concentration of the second protein (e.g., compB) by about 1.1, 1.2, 1.3, 1.4, 1.5, 2, 2.5, 3, 3.5, 4, 4.5, or 5-fold. In some embodiments, the molar concentration of the second protein (e.g., compB) is greater than the molar concentration of the first protein (e.g., compA). In some embodiments, the molar concentration of the second protein (e.g., compB) is greater than the molar concentration of the first protein (e.g., compA) by about 1.1, 1.2, 1.3, 1.4, 1.5, 2, 2.5, 3, 3.5, 4, 4.5, or 5-fold. In some embodiments, the molar concentration of the first protein (e.g., compA) is about 1-5 μM, 1-10 μM, 1-20 μM, 5-10 μM, 5-20 μM, 10-20 μM, 10-30 μM, 10-40 μM, or 10-50 μM. In some embodiments, the molar concentration of the first protein (e.g., compA) is about 50 μM, about 60 μM, about 70 μM, about 80 μM, about 90 μM, about 100 μM, about 200 μM, about 300 μM, or about 500 μM. In some embodiments, the molar concentration of the second protein (e.g., compB) is about 1-5 μM, 1-10 μM, 1-20 μM, 5-10 μM, 5-20 μM, 10-20 μM, 10-30 μM, 10-40 μM, or 10-50 μM. In some embodiments, the molar concentration of the second protein (e.g., compB) is about 50 μM, about 60 μM, about 70 μM, about 80 μM, about 90 μM, about 100 μM, about 200 μM, about 300 μM, or about 500 μM In some embodiments, the molar concentration of the first protein (e.g., compA) is about 1-20 μM and the molar concentration of the second protein (e.g., compB) is about 1-20 μM. In some embodiments, the molar concentration of the first protein (e.g., compA) is about 10 μM and the molar concentration of the second protein (e.g., compB) is about 8 μM. In some embodiments, the molar concentration of the first protein (e.g., compA) is about 100-500 μM and the molar concentration of the second protein (e.g., compB) is about 100-500 μM.

In some embodiments, the nanostructures of the disclosure are prepared using a micromixer chip such as, but not limited to, those from Harvard Apparatus (Holliston, Mass.), Dolomite Microfluidics (Royston, UK), Precision Nanosystems (Vancouver, BC), or Cytiva (Marlborough, Mass.). In some embodiments, the nanostructures of the disclosure are prepared using a micromixer chip as described in US20200023358, which is hereby incorporated by reference. In some embodiments, the nanostructures of the disclosure are prepared using a microfluidic mixing instrument based on the NanoAssembler platform (Precision Nanosystems (Vancouver, BC); Cytiva (Marlborough, Mass.)). In some embodiments, the nanostructures of the disclosure are prepared using a NanoAssembler Ignite system.

In some embodiments, the method of mixing, blending, or combining the first protein (e.g., compA) and the second protein (e.g., compB) under conditions that minimize shear stress (e.g., microfluidic mixing) results in improved yield of nanostructures comprising the first and second proteins (e.g., pbVLPs) as compared to mechanical mixing (e.g., using a blade, stir bar, or impeller) of the first and second protein. Methods for measuring yield of nanostructures (e.g., pbVLPs) is known in the art and includes quantifying the weight percent of total protein formed into nanostructure using, e.g., size exclusion chromatograph (SEC). In some embodiments, the method of mixing, blending, or combining the first protein (e.g., compA) and the second protein (e.g., compB) under conditions that minimize shear stress results in a yield of nanostructure (e.g., pbVLP) that is at least about 30%, about 40%, about 50%, about 60%, about 70%, or higher of total protein as measured by SEC. In some embodiments, the nanostructure is formed at a weight percent that is about 40% of total protein as measured by SEC. In some embodiments, the nanostructure is formed at a weight percent that is about 45% of total protein as measured by SEC. In some embodiments, the nanostructure is formed at a weight percent that is about 55% of total protein as measured by SEC. In some embodiments, the nanostructure is formed at a weight percent that is about 60% of total protein as measured by SEC. In some embodiments, the nanostructure is formed at a weight percent that is about 70%, about 75%, about 80%, about 85%, about 90%, or about 95% of total protein as measured by SEC.

In some embodiments, the method of mixing, blending, or combining the first protein (e.g., compA) and the second protein (e.g., compB) under conditions that minimize shear stress (e.g., microfluidic mixing) results in formation of nanostructures comprising the first and second proteins (e.g., pbVLPs) having a reduced polydispersity index as compared to mechanical mixing (e.g., using a blade, stir bar, or impeller) of the first and second protein. Methods for measuring the polydispersity of nanostructures are known in the art, and include SEC, light scattering (e.g., dynamic light scattering), and transmission electron microscopy (TEM). In some embodiments, the method of mixing, blending, or combining the first protein (e.g., compA) and the second protein (e.g., compB) under conditions that minimize shear stress results in formation of nanostructures having a polydispersity index that is less than about 0.4, about 0.35, about 0.3, about 0.25, about 0.2, about 0.15, or about 0.1.

### Methods of Purifying Nanostructures

The present disclosure provides methods for purifying nanostructures described herein (e.g., pbVLP particles) to remove excess soluble proteins (e.g., compA and/or compB) and/or impurities. As described herein, it was discovered that passing a mixture of pbVLPs through a large pore (e.g., about 1,000 kDa) filter with a hydrophilic membrane (e.g., regenerated cellulose (CRC) membrane) provided a solution of pbVLPs with a high degree of purity (e.g., >90%), without substantially reducing the yield of pbVLPs. In contrast, it was found that passing a mixture of pbVLPs through a large pore filter with a hydrophobic membrane (e.g., Pellicon Biomax 1000 kDa) or use of an alternative purification method (e.g., a Butyl Convective Interaction Media (CIM) column or a Captocore700 column) substantially diminished the yield of pbVLPs.

Accordingly, in some embodiments, the disclosure provides methods for purifying nanostructures described herein (e.g., pbVLPs) by filtration through a large pore hydrophilic membrane. In some embodiments, the filtering is performed by tangential flow filtration (TFF). In some embodiments, the hydrophilic membrane has a pore size of about 1,000 kDa. In some embodiments, the membrane has a pore size of about 300 kDa, about 400 kDa, about 500 kDa, about 600 kDa, about 700 kDa, about 800 kDa, or about 900 kDa. In some embodiments, the membrane has a pore size greater than about 1,000 kDa. In some embodiments, the membrane has a pore size of about 1100 kDa, about 1200 kDa, about 1300 kDa, about 1400 kDa, about 1500 kDa, about 2000 kDa, about 2500 kDa, or about 3000 kDa. In some embodiments, the hydrophilic membrane comprises a material selected from: cellulose, CRC, polytetrafluoroethylene (PTFE), polyvinylidene fluoride (PVDF), nylon, and polyethersulfone. In some embodiments, the large pore hydrophilic membrane is a CRC membrane with a pore size of about 1,000 kDa. In some embodiments, the large pore hydrophilic membrane is a Ultracel® 1000 kDa Membrane or an equivalent thereof.

In some embodiments, the purifying provides nanostructures (e.g., pbVLPs) with high purity, e.g., having minimal excess soluble protein (e.g., CompA and/or CompB), and/or impurities as measured by SEC. In some embodiments, the purity is measured as the percentage of total protein in the filtered solution that is formed into nanostructures, e.g., using SEC. In some embodiments, the purity is at least about 80%, 85%, 90%, 91%, 92%, 93%, 94%, 95%, 96%, 97%, 98%, or >99%. In some embodiments, the purifying provides the nanostructures (e.g., pbVLPs) without substantial loss relative to the mixture of nanostructures prior to the filtration, e.g., as measured by SEC. In some embodiments, at least about 80%, 85%, 90%, 91%, 92%, 93%, 94%, 95%, 96%, 97%, 98%, or >99% of nanostructures (e.g., pbVLPs) in the mixture prior to the filtration are recovered following passing through the large pore hydrophilic membrane, e.g., as measured by SEC.

### Exemplary Methods of Making Nanostructures

In some embodiments, the disclosure provides a method of making pbVLPs comprising compA and compB proteins, the method comprising (i) addition of compA protein to a solution comprising compB protein, optionally as compA emerges from the last step in its purification process (e.g., filtration); and (ii) mixing, blending, or combining compA under conditions that minimize shear stress, e.g., as compared to mechanical mixing of compA and compB, thereby forming the pbVLP comprising compA and compB proteins. In some embodiments, the conditions of (ii) comprising mixing the solution without mechanical mixing (e.g., using a blade, stir bar, or impeller). In some embodiments the conditions of (ii) comprise orbital agitation or microfluidic mixing. In some embodiments, the conditions of (ii) result in a partial or substantially homogenous mixture of compA and compB that facilitates assembly of compA and compB to form pbVLPs.

In some embodiments, the method comprises (i) addition of compA protein to a solution comprising compB protein, optionally as compA emerges from the last step in its purification process (e.g., filtration); and (ii) mixing, blending, or combining the compA and compB proteins by orbital agitation, thereby forming pbVLPs comprising compA and compB proteins. In some embodiments, the method comprises (i) addition of compA protein to a solution comprising compB protein, optionally as compA emerges from the last step in its purification process (e.g., filtration); (ii) providing conditions for mixing, blending, or combining of the solution by orbital agitation to form the pbVLPs; and (iii) purifying the pbVLPs to remove excess compA, excess compB, and/or impurities comprising filtering the solution with a large pore hydrophilic membrane by TFF. In some embodiments, the orbital agitation of (ii) comprises use of an orbital shaking platform. In some embodiments, the orbital agitation of (ii) is performed at about 20-25° C. In some embodiments, the orbital agitation of (ii) is performed for a duration between about 6 to about 24 hours. In some embodiments the orbital agitation of (ii) is performed for a duration between about 8 to about 12 hours. In some embodiments, the orbital agitation of (ii) is performed at a speed of about 80 to about 100 rpm. In some embodiments, the large pore hydrophilic membrane of (iii) is a CRC membrane comprising a pore size of about 300-3000 kDa, about 800 kDa, about 900 kDa, about 1,000 kDa, about 1100 kDa, about 1200 kDa, about 13 kDa, about 1400 kDa, or about 1500 kDa. In some embodiments, the large pore hydrophilic membrane of (iii) is a CRC membrane comprising a pore size of about 1000 kDa. In some embodiments, the large pore hydrophilic membrane of (iii) is a 1,000 kDa membrane is Ultracel® 1000 kDa Membrane or an equivalent thereof. In some embodiments, the solution comprising compB protein is prepared from one or more frozen batches of compB protein. In some embodiments, the steps of (i) and (ii) are a continuous process. In some embodiments, the steps of (i), (ii), and (iii), if present, are a continuous process. In some embodiments, compA protein is continuously produced prior to the addition of (i).

In some embodiments, the method of making pbVLPs comprising compA proteins and compB proteins comprises a microfluidic device. In some embodiments, the method comprises (i) providing a first inlet fluid stream comprising compA protein and a second inlet fluid stream comprising compB protein, and (ii) contacting the first inlet fluid stream and the second inlet fluid stream to form an outlet stream, wherein mixing, blending, or combining of the compA protein and the compB protein occurs in the outlet stream, thereby forming the pbVLP.

In some embodiments, the method comprises (i) providing a first inlet fluid stream comprising compA that flows through a first channel of a microfluidic device and a second inlet fluid stream comprising compB protein that flows through a second channel of the microfluidic device, and (iii) contacting the first inlet fluid stream and the second inlet fluid stream to form an outlet stream that flows through a third channel of the microfluidic device, wherein the first channel, second channel, and third channel are connected at an overlap region, e.g., having a T-shaped or Y-shaped configuration, and wherein the contacting of the first inlet fluid stream with the second inlet fluid stream enables mixing, blending, or combining of compA and compB, thereby resulting in formation of the pbVLPs. In some embodiments, the mixing, blending, or combining is facilitated by one or more passive diffusion elements present in the outlet stream, e.g., as splitting and recombination channels, grooves, slanted wells, or ridges. In some embodiments, the microfluidic device is a NanoAssemblr Ignite cartridge. In some embodiments, the method further comprises purifying the pbVLPs to remove excess compA, excess compB, and/or impurities comprising filtering the solution with a large pore hydrophilic membrane by TFF. In some embodiments, the large pore hydrophilic membrane of is a CRC membrane comprising a pore size of about 300-3000 kDa, about 800 kDa, about 900 kDa, about 1,000 kDa, about 1100 kDa, about 1200 kDa, about 13 kDa, about 1400 kDa, or about 1500 kDa. In some embodiments, the large pore hydrophilic membrane is a CRC membrane comprising a pore size of about 1000 kDa. In some embodiments, the large pore hydrophilic membrane of is a 1,000 kDa membrane is Ultracel® 1000 kDa Membrane or an equivalent thereof. In some embodiments, the flow path of the outlet stream feeds directly into the TFF, thereby providing a continuous process for production and purification of the pbVLPs. In some embodiments, the compA protein is continuously produced prior to introduction to the first inlet fluid stream.

In some embodiments, the method provides a substantially pure composition of pbVLPs comprising compA and compB, e.g., having minimal excess soluble protein (e.g., CompA and/or CompB), and/or impurities as measured by SEC. In some embodiments, the purity of the pbVLPs is determined as the weight percent of total protein in the purified solution that is pbVLPs as measured by SEC. In some embodiments, at least about 80%, 85%, 90%, 91%, 92%, 93%, 94%, 95%, 96%, 97%, 98%, or >99% of total protein in the purified solution is pbVLPs.

In some embodiments, the compA protein is a dimer described herein. In some embodiments, the compA protein is a trimer described herein. In some embodiments, the compA protein is a pentamer described herein. In some embodiments, the compA protein comprises a polypeptide sequence having at least about 80%, 85%, 90%, 91%, 92%, 93%, 94%, 95%, 96%, 97%, 98%, 99%, or 100% sequence identity to any one of SEQ IN NOs: 1, 3, 5, 7, 9, 11, 13, 15, 17, 19, 21, 23, 25, 26, 29-31, 35, 37, 39, 41, 43, 45, 47, 49, 51, 53, 55, 57, and 59. In some embodiments, the compB protein is a dimer described herein. In some embodiments, the compB protein is a trimer described herein. In some embodiments, the compB protein is a pentamer described herein. In some embodiments, the compB protein comprises a polypeptide sequence having at least about 80%, 85%, 90%, 91%, 92%, 93%, 94%, 95%, 96%, 97%, 98%, 99%, or 100% sequence identity to any one of SEQ IN NOs: 2, 4, 6, 8, 10, 12, 14, 16, 18, 20, 22, 24, 27, 28, 32-34, 36, 38, 40, 42, 44, 46, 48, 50, 52, 54, 56, and 58.

In some embodiments, the compA protein is a trimer and the compB protein is a pentamer. In some embodiments, the compA protein and the compB protein each comprises a polypeptide sequence having at least about 80%, 85%, 90%, 91%, 92%, 93%, 94%, 95%, 96%, 97%, 98%, 99%, or 100% sequence identity to SEQ ID NOS: 39 and 40 respectively. In some embodiments, the compA protein comprises a polypeptide sequence having at least about 80%, 85%, 90%, 91%, 92%, 93%, 94%, 95%, 96%, 97%, 98%, 99%, or 100% sequence identity to SEQ ID NO: 7 and the compB protein comprises a polypeptide sequence having at least about 80%, 85%, 90%, 91%, 92%, 93%, 94%, 95%, 96%, 97%, 98%, 99%, or 100% sequence identity to any one of SEQ ID NOS: 8, 32, 33, and 34. In some embodiments, the compA protein comprises a polypeptide sequence having at least about 80%, 85%, 90%, 91%, 92%, 93%, 94%, 95%, 96%, 97%, 98%, 99%, or 100% sequence identity to SEQ ID NO: 29 and the compB protein comprises a polypeptide sequence having at least about 80%, 85%, 90%, 91%, 92%, 93%, 94%, 95%, 96%, 97%, 98%, 99%, or 100% sequence identity to any one of SEQ ID NOS: 8, 32, 33, and 34. In some embodiments, the compA protein comprises a polypeptide sequence having at least about 80%, 85%, 90%, 91%, 92%, 93%, 94%, 95%, 96%, 97%, 98%, 99%, or 100% sequence identity to SEQ ID NO: 30 and the compB protein comprises a polypeptide sequence having at least about 80%, 85%, 90%, 91%, 92%, 93%, 94%, 95%, 96%, 97%, 98%, 99%, or 100% sequence identity to any one of SEQ ID NOS: 8, 32, 33, and 34. In some embodiments, the compA protein comprises a polypeptide sequence having at least about 80%, 85%, 90%, 91%, 92%, 93%, 94%, 95%, 96%, 97%, 98%, 99%, or 100% sequence identity to SEQ ID NO: 31 and the compB protein comprises a polypeptide sequence having at least about 80%, 85%, 90%, 91%, 92%, 93%, 94%, 95%, 96%, 97%, 98%, 99%, or 100% sequence identity to any one of SEQ ID NOS: 8, 32, 33, and 34.

### Nucleic Acids

In another aspect, the present disclosure provides isolated nucleic acids encoding an antigen, a first component, and/or a second component, of the present disclosure. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

In a further aspect, the present disclosure provides recombinant expression vectors comprising the isolated nucleic acid of any embodiment or combination of embodiments of the disclosure operatively linked a suitable control sequence. “Recombinant expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the host organisms either as an episome or by integration into host chromosomal DNA. In a preferred embodiment, the expression vector comprises a plasmid. However, the disclosure is intended to include other expression vectors that serve equivalent functions, such as viral vectors.

In another aspect, the present disclosure provides host cells that have been transfected or transduced with the recombinant expression vectors disclosed herein, wherein the host cells can be either prokaryotic or eukaryotic. The cells can be transiently or stably transfected or transduced. Such transfection or transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R. I. Freshney. 1987. Liss, Inc. New York, N.Y.).

In another aspect, the disclosure provides a method of producing an antigen, component, or pbVLP according to the disclosure. In some embodiments, the method comprises the steps of (a) culturing a host according to this aspect of the disclosure under conditions conducive to the expression of the polypeptide, and (b) optionally, recovering the expressed polypeptide.

In some embodiments, the disclosure provides a method of manufacturing a vaccine, comprising culturing a host cell comprising a polynucleotide comprising a sequence encoding the antigen of the disclosure in a culture medium so that the host cell secretes the antigen into the culture media; optionally purifying the antigen from the culture media; mixing the antigen with a second component, wherein the second component multrimerizes with the antigen to form a pbVLP; and optionally purifying the pbVLP.

In some embodiments, the disclosure provides method of manufacturing a vaccine, comprising culturing a host cell comprising one or more polynucleotides comprising sequences encoding both components of the pbVLP of any one of disclosure so that the host cell secretes the first component and the second component into the culture media; and optionally purifying the pbVLP from the culture media.

Illustrative host cells in include E. coli cells, 293 and 293F cells, HEK293 cells, Sf9 cells, Chinese hamster ovary (CHO) cells and any other cell line used in the production of recombinant proteins.

### Formulation

In some embodiments, the buffer in the composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the VLP, in lyophilized or liquid form. Exemplary stabilizers include sucrose, sorbitol, glycine, inositol, sodium chloride, methionine, arginine, and arginine hydrochloride.

### Methods of Use

In some embodiments, the disclosure provides methods for treating or preventing a disease or disorder in a subject in need thereof comprising administering a nanostructure (e.g., pbVLP) prepared according to a method described herein. In some embodiments, the disclosure provides a method for inducing, promoting, or increasing an immune response in a subject in need thereof, comprising administering a nanostructure (e.g., pbVLP) prepared according to a method described. In some embodiments, the nanostructure comprises one or more antigens. In some embodiments, the one or more antigens are displayed on the surface of the nanostructure. In some embodiments, the nanostructure comprises one or more immunostimulatory molecules attached to the exterior and/or encapsulated in the cage interior. As used herein, an immunostimulatory molecules is a compound that stimulates an immune response (including enhancing a pre-existing immune response) in a subject to whom it is administered, whether alone or in combination with another agent (e.g., an antigen). Exemplary immunostimulatory molecules include, but are not limited to, TLR ligands.

In some embodiments, the disclosure provides a method for inducing, promoting, or increasing an immune response in a subject in need thereof, comprising administering a nanostructure (e.g., pbVLP) prepared according to a method described herein as an immunogenic composition or vaccine. Upon introduction into a host, the immunogenic composition or vaccine provokes an immune response. The “immune response” refers to a response that induces, increases, or perpetuates the activation or efficiency of innate or adaptive immunity. In some embodiments, the immune response comprises production of antibodies and/or cytokines. In some embodiments, the immune response comprises activation of cytotoxic T cells, antigen presenting cells, helper T cells, dendritic cells, B cells, and/or other cellular responses.

In some embodiments, the disclosure provides a method for inducing, promoting, or increasing an antibody response in a subject in need thereof, comprising administering a nanostructure (e.g., pbVLP) prepared according to a method described herein as an immunogenic composition or vaccine, wherein the nanostructure displays one or more antigens, and wherein the antibody response is directed to epitopes present on the one or more antigens. Methods for analyzing an antibody response in a subject are known to those of skill in the art. For example, in some embodiments, an increase in an immune response is measured by ELISA to determine antigen-specific antibody titers.

In some embodiments, the disclosure provides a method for inducing, promoting, or increasing an immune response comprising an improved B-memory cell response in a subject in need thereof, comprising administering a nanostructure (e.g., pbVLP) prepared according to a method described herein as an immunogenic composition or vaccine in immunized subjects. An improved B-memory cell response is intended to mean an increased frequency of peripheral blood B cells capable of differentiation into antibody-secreting plasma cells upon antigen encounter as measured by stimulation of in vitro differentiation. In some embodiments, the disclosure provides methods for increasing the number of antibody-secreting B cells. In some embodiments, the antibody-secreting B cells are bone marrow plasma cells or germinal B cells. In some embodiments, methods for measuring antibody secreting B cells includes, but is not limited to, antigen-specific ELISPOT assays and flow cytometry of plasma cells or germinal center B cells collected at various time points post-immunization.

In some embodiments, the nanostructure (e.g., pbVLP) is administered as part of a prophylactic immunogenic composition or vaccine, wherein the immunogenic composition or vaccine confers resistance in a subject to subsequent exposure to infectious agents. In some embodiments, the nanostructure (e.g., pbVLP) is administered as part of a therapeutic immunogenic composition or vaccine, wherein the immunogenic composition or vaccine initiates or enhances a subject's immune response to a pre-existing antigen. In some embodiments, the pre-existing antigen is a viral antigen in a subject infected with an infectious agent or neoplasm. In some embodiments, the pre-existing antigen is a cancer antigen in a subject with a tumor or malignancy. The desired outcome of a prophylactic or therapeutic immune response depends upon the disease or condition being treated, according to principles well known in the art. For example, in some embodiments, an immune response against an infectious agent may completely prevent colonization and replication of an infectious agent. In some embodiments, a vaccine against infectious agents is considered effective if it reduces the number, severity, or duration of symptoms, if it reduces the number of individuals in a population with symptoms, or reduces the transmission of an infectious agent. In some embodiments, an immune response against cancer, allergens, or infectious agents is effective it completely treats a disease, alleviates symptoms, or contributes to an overall therapeutic intervention against a disease.

In some embodiments, the disclosure provides a method for treating or preventing an acute or chronic infectious disease in a subject in need thereof, comprising administering a nanostructure (e.g., pbVLP) prepared according to a method describe herein as an immunogenic composition or vaccine. In some embodiments, the nanostructure (e.g., pbVLP) comprises one or more infectious disease antigens. In some embodiments, the one or more infectious disease antigens is a microbial antigen. Microbial antigens are antigens derived from a microbial species, e.g., a bacteria, virus, fungus, parasite, or mycobacterium. In some embodiments, the disclosure provides a method for treating or preventing a viral infection in a subject in need thereof, comprising administering a nanostructure (e.g., pbVLP) prepared according to a method describe herein as an immunogenic composition or vaccine, wherein the nanostructure comprises one or more infectious disease antigens derived from the virus. In some embodiments, the viral infection is immunodeficiency (e.g., HIV, papilloma (e.g., HPV), herpes (e.g., HSV), encephalitis, influenza (e.g., human influenza virus A), COVID-19 (e.g., SARS-CoV-2), and common cold (e.g., human rhinovirus, respiratory syncytial virus). In some embodiments, the disclosure provides a method for reducing a viral infection in a subject in need thereof, comprising administering to the subject a nanostructure (e.g., pbVLP) prepared according to a method described herein.

In some embodiments, the disclosure provides a method for treating or preventing a disorder associated with abnormal apoptosis, a differentiation process (e.g., cellular proliferative disorders, e.g., hyperproliferative disorders), or a cellular differentiation disorder (e.g., cancer). Examples of cellular proliferative and/or differentiative disorders include cancer (e.g., carcinoma, carcinoma, metastatic disorders, or hematopoietic neoplastic disorders). In some embodiments, an immunogenic composition or vaccine comprising a nanostructure (e.g., pbVLP) prepared according to a method described herein is administered to a subject who has cancer. The term “cancer” refer to malignancies of the various organ systems, including those affecting the lung, breast, thyroid, lymphoid tissues, gastrointestinal organs, and the genitourinary tract, as well as to adenocarcinomas that are generally considered to include malignancies such as most colon cancers, renal-cell carcinomas, prostate cancer and/or testicular tumors, non-small cell carcinoma of the lung, cancer of the small intestine, and cancer of the esophagus. In some embodiments, the immunogenic composition or vaccine is used to treat a subject who has, who is suspected of having, or who may be at high risk for developing any type of cancer.

In some embodiments, the disclosure provides methods for inducing an anti-tumor immune response in a subject with cancer, comprising administering an immunogenic composition or vaccine comprising a nanostructure (e.g., pbVLP) prepared according to a method described herein. In some embodiments, the nanostructure comprises one or more antigens, wherein the antigens are cancer antigens. A cancer antigen is an antigen that is expressed preferably on cancer cells (i.e., it is expressed at higher levels in cancer cells than on non-cancer cells) and in some instances is solely expressed by cancer cells. The cancer antigen may be expressed within a cancer cell or on the surface of the cancer cell. In some embodiments, administering the immunogenic composition or vaccine comprising a nanostructure (e.g., pbVLP) comprising one or more cancer antigens induces an anti-tumor immune response, thereby preventing or treating a cancer in the subject.

### EXAMPLES

### Example 1

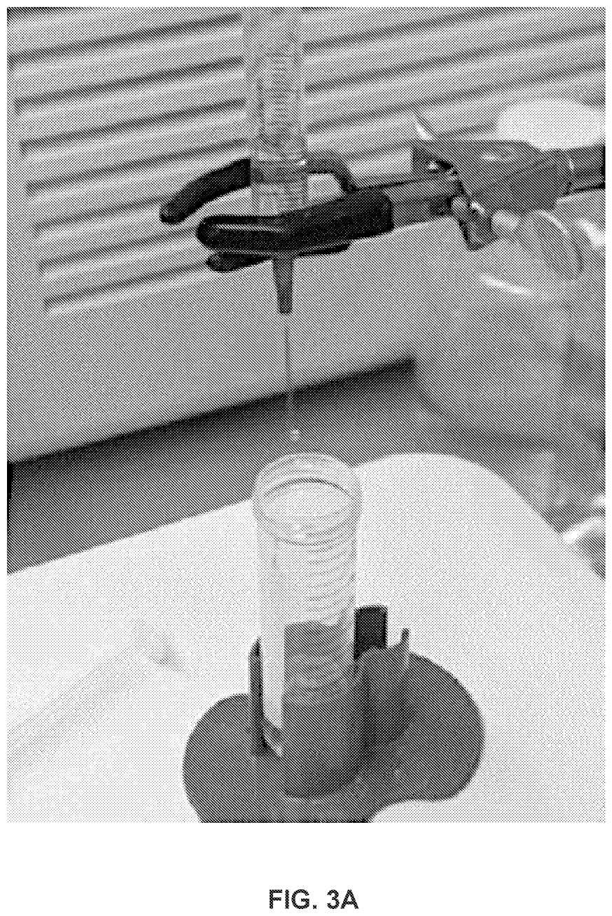

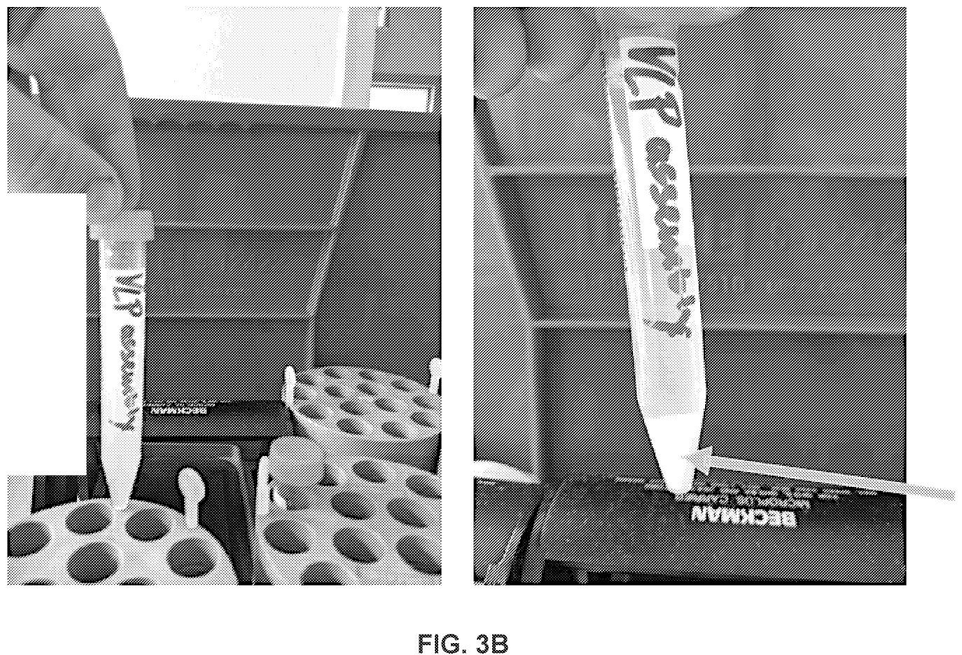

This example demonstrates improvement in the assembly process for a two-component nanostructure by employing slow addition of CompB to CompA under the surface of the CompA solution. Purified component A (compA) protein (I53-50A) fused to RSV antigen and purified component B (compB) protein (I53-50B) were separately purified into assembly buffer. The compA sample was added dropwise from a 22 G ½ needle at about 0.6 mL/min to the compB sample under stirring at 100 rpm with a magnetic stir bar over about 10 minutes, to a final molar ratio of 1:1 compA:compB. The experimentation set up is shown in FIG. 3A. A turbid solution was formed (FIG. 3B, left). The pellet (FIG. 3B, right) did not, however contain the compA:compB assembly, which remained in solution.

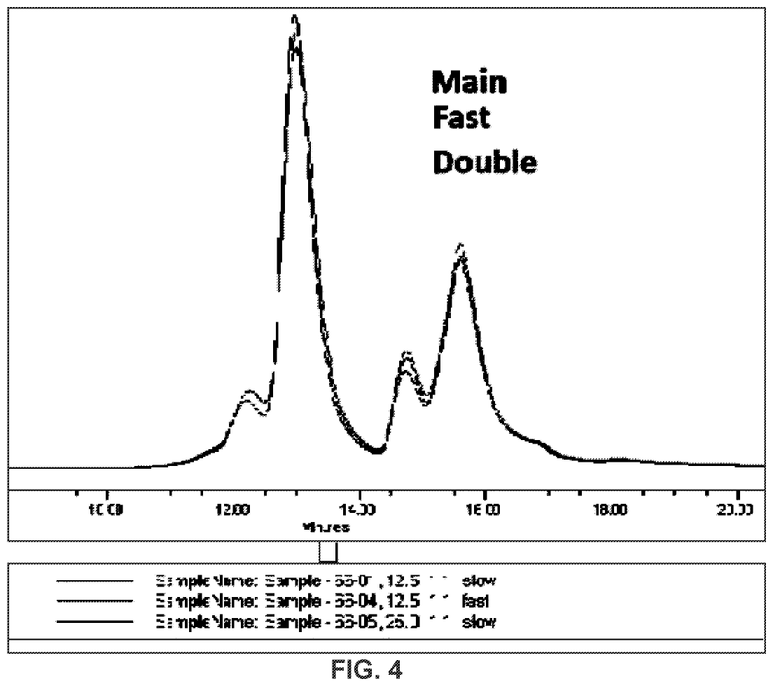

Next, the same assembly reaction was performed using a serological pipette slowly dispensing 2504 CompA into 25 μM CompB (about 30 mL volume per each) under the surface of the CompB solution at about 20 mL/min, without a stir bar. After addition of the CompA solution into the CompB solution, the mixture was placed only an orbital agitation platform overnight at 80 rpm at room temperature. Slight turbidity was removed by centrifugation at 2000×g for five minutes. As shown in the bottom panel of FIG. 4, high performance liquid chromatograph (HPLC) analysis demonstrated formation of the VLP assembly (50%) with minor peaks for high molecular weight (HMW) aggregate (7.6%), excess CompA (9.5%) and other impurities (31.6%).

In conclusion, mixing with a stir bar placed into the solution during assembly led to reduced product quality (assessed by SE-HPLC). Mixing the solution by orbital agitation platform improved product quality.

### Example 2

This example demonstrates improvement in the assembly process for a two-component nanostructure by employing a 1000 kDa molecule weight cut-off membrane composed of composite regenerated cellulose (Pellicon Ultracel 1000 kDa) to separate pbVLP from lower molecular weight impurities. The crude pbVLP mixture was prepared by dispensing a solution of CompA fused to RSV into a solution of CompB, at a molar ratio of 1:1, followed by orbital agitation as described in Example 1. The pbVLP is purified from 45% w/w total protein to 97% w/w total protein (2×purification) with a yield of 95%. In contrast, other purification strategies exhibited poor yield. Purification strategies having poor yield included hydrophobic (Pellicon Biomax 1000 kDa) membrane filtration (17% yield), Butyl CIM chromatography (0% yield) and Captocore700 chromatography. Data from Tangential Flow Filtration (TFF) using a Pellicon Biomax 1000 kDa membrane are summarized in Table 4 below. The crude pbVLP mixture loaded for filtration (“TFF Load”) had a total protein concentration of 1.18 mg/mL and contained 70.8 mg total protein. The crude pbVLP was filtered using a diafiltrate volume that was either 10-fold or 5-fold greater than the load volume. The purity and yield of the pbVLP was high for both conditions.

TABLE 4
Tangential Flow Filtration using a Pellicon Ultracel 1000 kDa Membrane

| | | Total | Re- | | | |
|---|---|---|---|---|---|---|
| | Concentration | protein | covery | Purity | Amount | Yield |
| Fractions | (mg/mL) | (mg) | (%) | (%) | (mg) | (%) |
| TFF Load | 1.18 | 70.8 | 100 | 45 | 31.86 | 100 |
| TFF Retentate, | 0.52 | 31.2 | 44.1 | 95.3 | 29.73 | 93.3 |
| after 10 × | | | | | | |
| volume | | | | | | |
| diafiltration for | | | | | | |
| purification | | | | | | |
| TFF Retentate, | 1.46 | 29.2 | 93.6 | 96.8 | 28.27 | 95.1 |
| after 5 × | | | | | | |
| diafiltration to | | | | | | |
| exchange into | | | | | | |
| formulation | | | | | | |
| buffer | | | | | | |

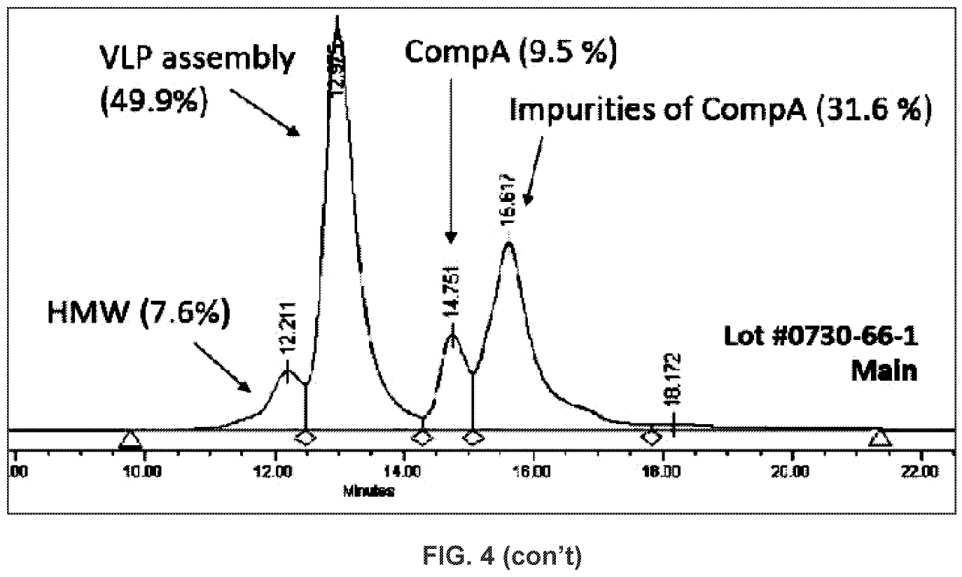

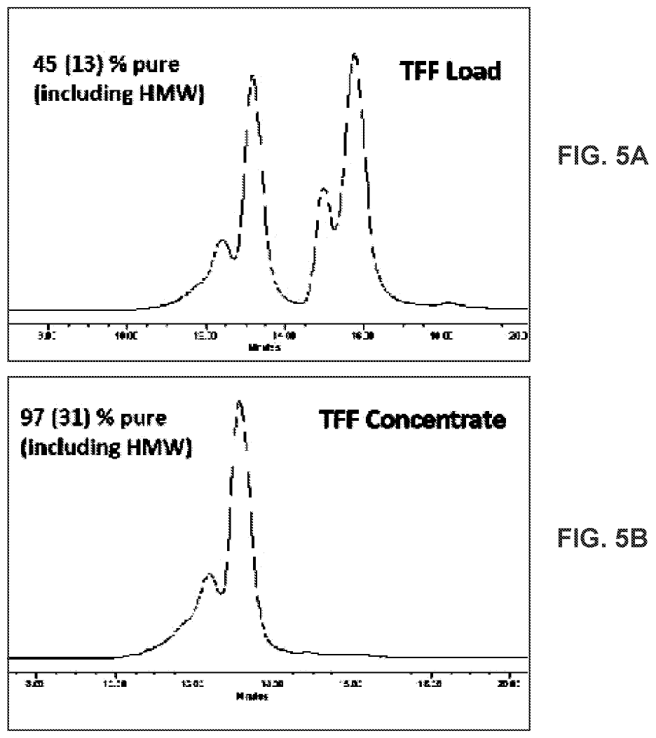

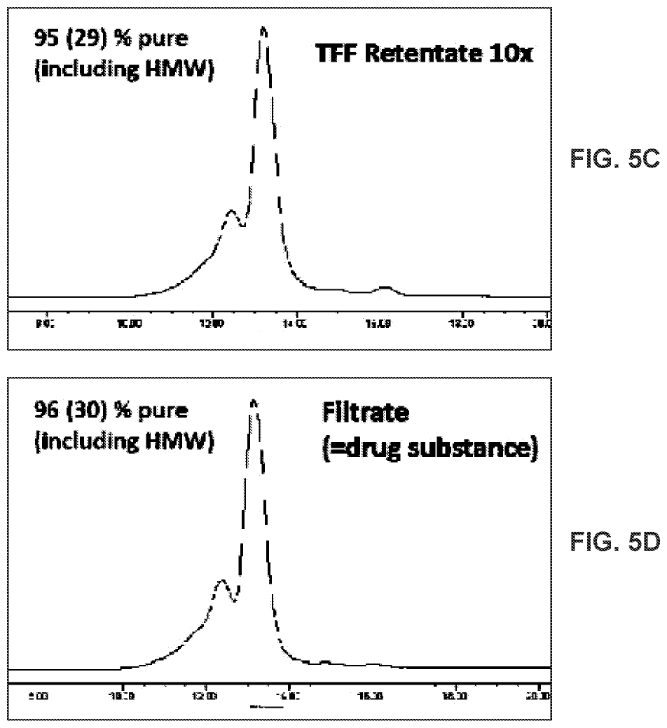

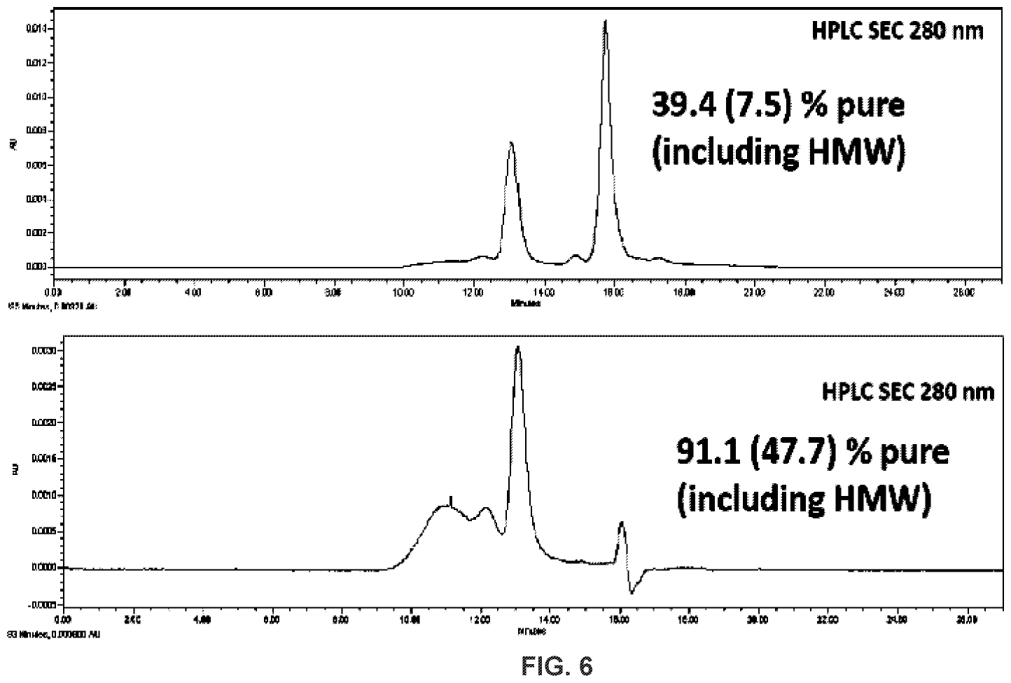

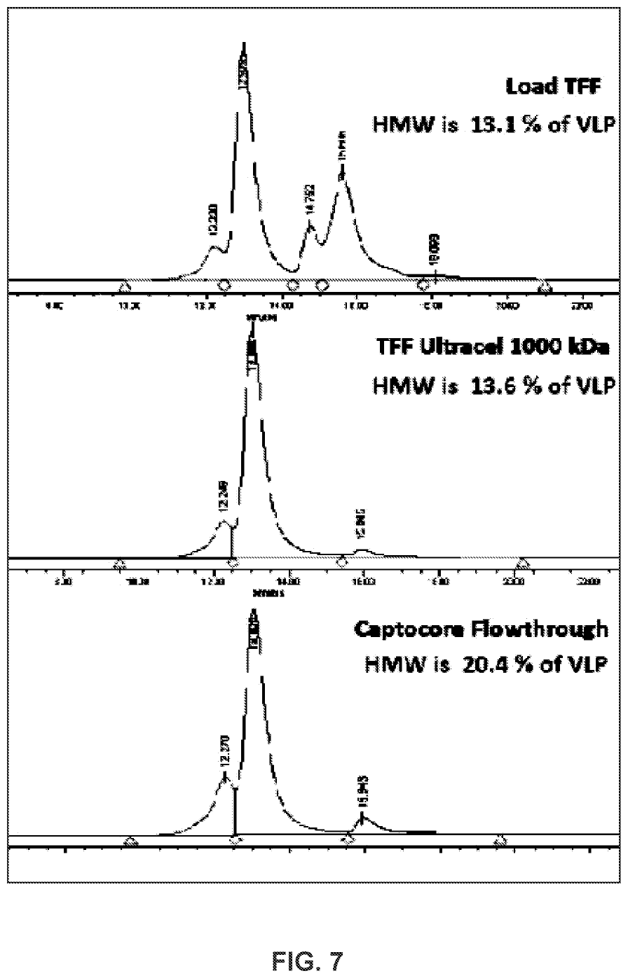

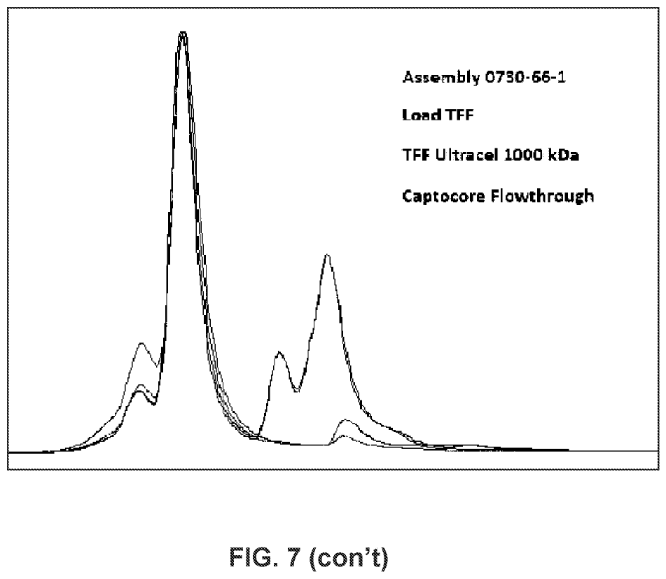

In conclusion, contacting the pbVLP assembly with a 1,000 kilodalton molecular weight cut-off membrane to purify the pbVLP increased purity without impacting product quality. Data shows the SE profile was not affected (FIG. 5). Several other purification methods degraded the SE profile (FIG. 6). Using Pellicon Biomax 1000 kDa to purify the VLP gave a yield of 17% and changed the SE-HPLC profile. Using a Pellicon Ultracel 1000 kDa membrane gives a nearly 100% yield.

### Example 3

Scalable assembly of virus-like-particles using the two-component CompA/CompB system requires unit operations that provide robust, homogenous and rapid mixing of input process solutions, and results in pbVLPs with the desired product quality attributes. Ideally, the assembly unit operation would additionally be amenable to continuous manufacturing configurations. The NanoAssemblr® Ignite™ system (Product code NIN0001) is a microfluidics mixing device that delivers process solutions at precise flow rates and ratios, and utilizes disposable cartridges containing a precisely defined mixing flowpath. The Ignite system is designed to provide a representative small-scale model to evaluate mixing operations that can be directly scaled up using process equipment that can be configured to allow continuous manufacturing.

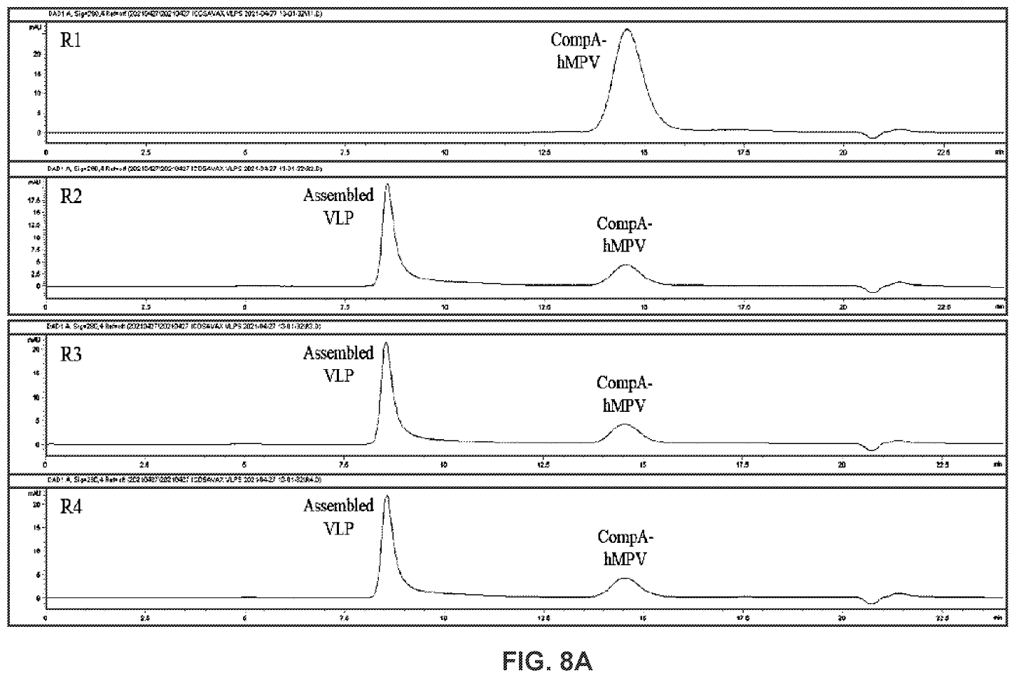

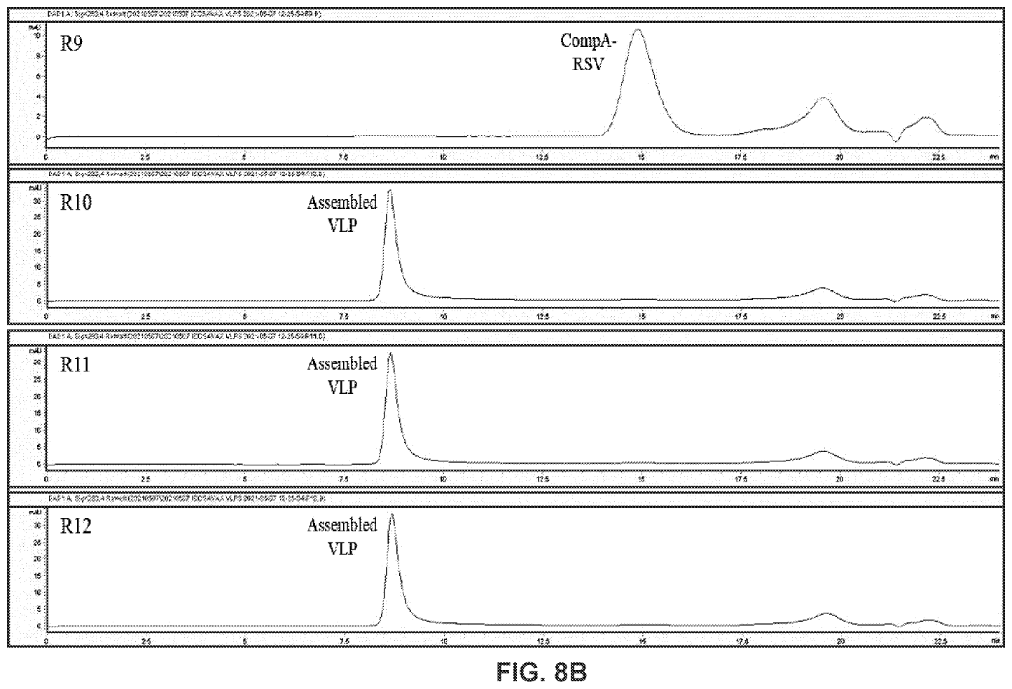

To evaluate pbVLP assembly using a microfluidics mixing approach, the Ignite system was used to assemble pbVLPs from a trimer of CompA (I53-50A) fused to an hMPV antigen having a his-tag (CompA-hMPV-his; homologous to SEQ ID NO: 60 shown in Table 3) and a CompB-01 pentamer (SEQ ID NO: 63 shown in Table 3). For VLP assembly using the Ignite microfluidics mixing system, the individual components were adjusted to defined concentrations (10 μm CompA/8 μm CompB) in assembly buffer (20 mM Tris, 250 mM sodium chloride, 5% glycerol, pH-7.4) and loaded into single-use 1 mL BD syringes. The loaded syringes were attached to distinct channels of single-use, disposable Ignite NxGen microfluidic cartridges and assembly reaction conducted by passing the stock solutions through cartridges at a 1:1 volume ratio and two different total system flow rates (2 mL/min and 10 mL/min). Standard assembly reactions were conducted by adding the stock solutions to microcentrifuge tubes at a 1:1 volume ratio using a pipette for addition of compA fusion to compB, followed by orbital shaking to achieve mixing. HPLC-SEC analysis was used to assess pbVLP assembly compared to a CompA control (10 uM CompA-hMPV-his; R1 in FIG. 8A). As shown in FIG. 8A, standard assembly (R2) and assembly by the Ignite microfluidic mixing system at either 2 mL/min (R3) or 10 mL/min (R4) yielded pbVLP.

Also evaluated was the use of the Ignite system to assemble pbVLP from a trimer of CompA (I53-50A) fused to an RSV antigen (CompA-RSV-02; SEQ ID NO: 61 shown in Table 3) and a CompB-01 pentamer (SEQ ID NO: 63 shown in Table 3). The assembly reaction was performed using the Ignite microfluidics mixing system as described above from a 10 μm stock solution of CompA-RSV-02 and a 8 μm stock solution of CompB. Standard assembly reactions were conducted by adding the stock solutions to microcentrifuge tubes at a 1:1 volume ratio using a pipette for addition and mixing. The VLP assembly reactions were evaluated by HPLC-SEC analysis and compared to CompA control (10 uM CompA-RSV-02; 0.5 mL total loading volume; R9 in FIG. 8B). As shown in FIG. 8B, the standard assembly (R10; 0.5 mL total loading volume), and assembly by the Ignite microfluidic mixing system at either 2 mL/min (R11; 1.0 mL total loading volume) or 10 mL/min (R12; 1.0 mL total loading volume) yielded assembled VLP.

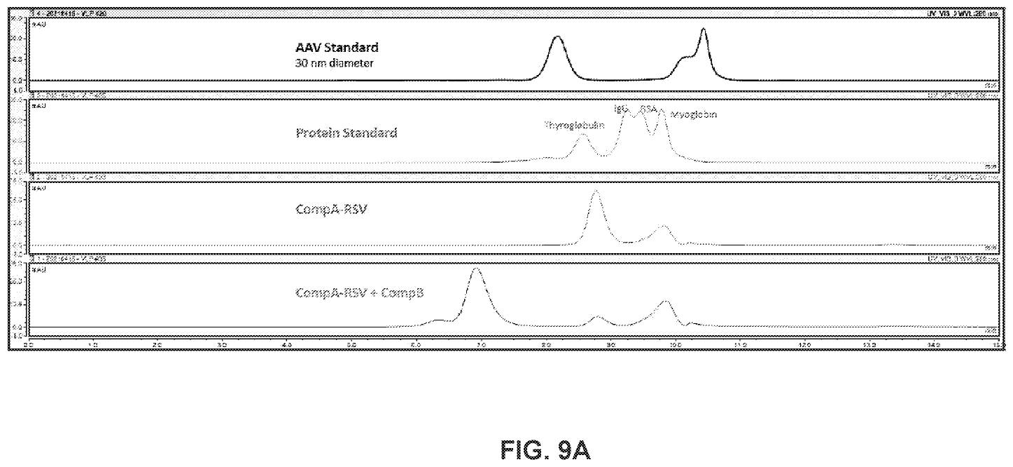

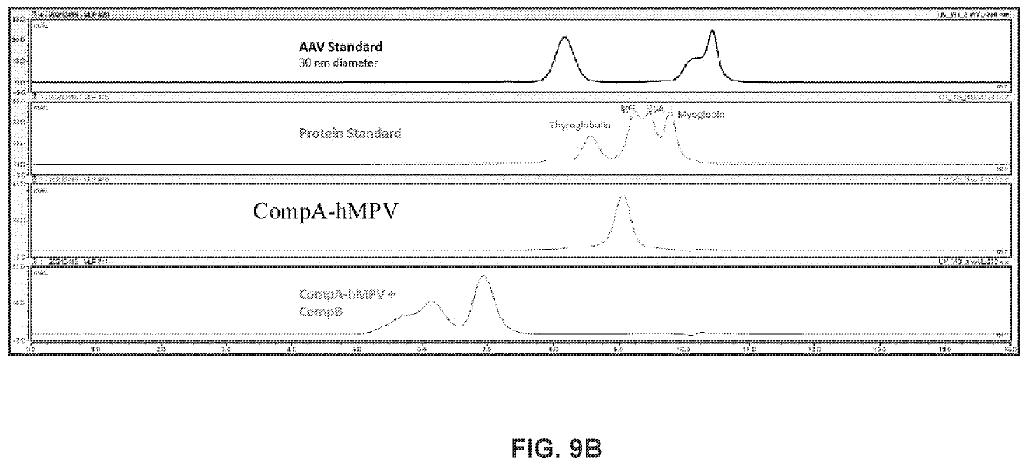

For purposes of comparison, pbVLP assembly was evaluated by simple mixing. Briefly, a 10 μM stock solutions of CompA-hMPV-his or CompA-RSV-02 was added by pipette to an 8 μM solution of CompB pentamer (153-50B) at a 1:1 volume. The solution was then mixed by orbital agitation and incubated at 4° C. for 2 hours prior to analysis. The resulting mixture was evaluated by HPLC-SEC (Agilent Bio SEC-5 1000 Å column; flow rate of 0.4 mL/min; mobile phase of 25 mM NaPi+300 mM NaCl, pH 6.6). Comparison was made to a standard that was an AAV capsid with 30 nm diameter, a set of protein standards (thyroglobulin (660 kDa), IgG (150 kDa), BSA (66 kDa), and myoglobin (17 kDa)), and respective CompA protein. As shown in FIG. 9A, simple mixing provided assembled pbVLP for the CompA-RSV fusion with good yield and uniformity. However, in this case as shown in FIG. 9B, simple mixing of CompA-hMPV-his+CompB resulted in a pbVLP mixture with substantial amount of high molecular weight aggregate. Variation in the pbVLP assembly using simple mixing (i.e., pipette addition of compA to compB with mixing via orbital agitation) is avoided using microfluidic mixing of the pbVLP components.

Together, these data demonstrate that efficient pbVLP assembly is achieved using a microfluids mixing system and indicates the utility of the approach for scale-up GMP manufacturing.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of making a nanostructure, comprising adding a component A (compA) protein to a solution comprising a component B (compB) protein under conditions that minimize shear stress, thereby forming a compA:compB complex.

2. The method of claim 1, wherein the conditions that minimize shear stress comprise adding the compA protein to the compB protein under the surface of the solution.

3. The method of claim 1 or claim 2, wherein the conditions that minimize shear stress comprise mixing the solution without mechanical mixing.

4. The method of any one of claims 1-3, wherein the conditions that minimize shear stress comprise mixing the solution without a stir bar.

5. The method of any one of claims 1-4, wherein the conditions that minimize shear stress comprise mixing the solution without an impeller.

6. The method of any one of claims 1-5, wherein the conditions that minimize shear stress comprise mixing the solution using an orbital agitation.

7. The method of any one of claims 1-6, wherein the conditions that minimize shear stress comprise mixing the solution using a microfluidic mixer.

8. The method of any one of claim 1-7, wherein the method comprising adding the compA protein in excess to the compB protein.

9. The method of any one of claims 1-8, wherein the conditions provide for mixing of compA and compB without substantial precipitation, optionally relative to mechanical mixing of a solution of compA and compB.

10. The method of any one of claims 1-9, wherein the compA:compB complex is formed in an amount that is at least about 40% of total protein, optionally as measured by size exclusion chromatography.

11. The method of any one of claims 1-10, wherein the method further comprises purifying the compA:compB complex from excess compA, excess compB, and/or other impurities by filtering the solution with a 1,000 kDa membrane or an equivalent thereof.

12. The method of claim 11, wherein the 1,000 kDa membrane is a composite regenerated cellulose (CRC) membrane.

13. The method of claim 12, wherein the 1,000 kDa membrane is Ultracel® 1000 kDa Membrane or an equivalent thereof.

14. The method of any one of claims 11-13, wherein the filtering comprises tangential flow filtration (TFF).

15. The method of any one of claims 11-14, wherein the purifying provides the compA:compB complex with purity of about 80% or higher as measured by percent weight of total protein.

16. The method of any one of claim 1-15, wherein the method does not comprise purifying the solution with a hydrophobic membrane (e.g., Pellicon Biomax 1000 kDa), a Butyl Convective Interaction Media (CIM) column, a Captocore700 column, or an equivalent thereto.

17. The method of any one of claims 1-16, wherein the method is a continuous or semi-continuous process.

18. The method of any one of claims 1-17, wherein the compA protein is continuously produced prior to the adding step.

19. The method of any one of claims 1-18, wherein the compB protein is provided as one or more frozen batches.

20. A method of making a nanostructure, comprising (i) providing a first inlet fluid stream comprising a first protein and a second inlet fluid stream comprising a second protein, and (ii) contacting the first inlet fluid stream and the second inlet fluid stream to form an outlet stream, wherein mixing of the first protein and the second protein occurs in the outlet stream, thereby forming a protein complex comprises the first protein and the second protein.

21. The method of claim 20, wherein the first inlet fluid stream and the second inlet fluid stream are joined in a three-way configuration to form the outlet stream, optionally wherein the three-way configuration is a T-shaped or Y-shaped configuration.

22. The method of claim 20 or 21, wherein the first inlet fluid stream and the second inlet fluid stream are joined to form the outlet stream using a microfluidic mixer, optionally wherein the microfluidic mixer is a Nanoassembler® Ignite™ cartridge or any equivalent thereof.

23. The method of claim 22, wherein the microfluidic mixer comprises one or more passive mixing elements to facilitate mixing of the first protein and the second protein.

24. The method of any one of claims 20-23, wherein the outlet stream comprises a molar concentration of the first protein that exceeds the molar concentration of the second protein.

25. The method of any one of claims 20-23, wherein the outlet stream comprises a molar concentration of the first protein that is substantially equivalent to the molar concentration of the second protein.

26. The method of any one of claims 20-25, wherein the mixing of the first protein and the second protein occurs without substantial precipitation of the first protein, the second protein, the complex, or a combination thereof.

27. The method of any one of claims 20-26, wherein the complex is formed in an amount that is at least about 40% of total protein, optionally as measured by size exclusion chromatography.

28. The method of any one of claims 20-27, wherein the method further comprises purifying the complex from excess second protein, and/or other impurities by filtering the solution with a 1,000 kDa membrane or an equivalent thereof.

29. The method of claim 28, wherein the 1,000 kDa membrane is a composite regenerated cellulose (CRC) membrane.

30. The method of claim 29, wherein the 1,000 kDa membrane is Ultracel® 1000 kDa Membrane or an equivalent thereof.

31. The method of any one of claims 28-30, wherein the filtering comprises tangential flow filtration (TFF).

32. The method of any one of claims 28-31, wherein the purifying provides the complex with purity of about 90% or higher as measured by percent weight of total protein.

33. The method of any one of claim 20-32, wherein the method does not comprise purifying the solution with a hydrophobic membrane (e.g., Pellicon Biomax 1000 kDa), a Butyl Convective Interaction Media (CIM) column, a Captocore700 column, or an equivalent thereto.

34. The method of any one of claims 20-33, wherein the method is a continuous or semi-continuous process.

35. The method of any one of claims 20-34, wherein the first protein is continuously produced prior to the adding step.

36. The method of any one of claims 20-35, wherein the second protein is provided as one or more frozen batches.

37. The method of any one of claims 20-36, wherein the first component is a component A (compA) and/or the first component is a component B (compB).

38. The method of any one of claim 1-19 or 37, wherein the compA comprises a polypeptide sequence that has at least 90%, at least 95%, at least 99%, or 100% identity to any one of SEQ IN NOs: 1, 3, 5, 7, 9, 11, 13, 15, 17, 19, 21, 23, 25, 26, 29-31, 35, 37, 39, 41, 43, 45, 47, 49, 51, 53, 55, 57, and 59.

39. The method of any one of claim 1-19 or 37-38, wherein the compB comprises a polypeptide sequence that has at least 90%, at least 95%, at least 99%, or 100% identity to any one of SEQ IN NOs: 2, 4, 6, 8, 10, 12, 14, 16, 18, 20, 22, 24, 27, 28, 32-34, 36, 38, 40, 42, 44, 46, 48, 50, 52, 54, 56, and 58.

40. The method of any one of claim 1-19, or 37, wherein the compA and the compB each comprise a polypeptide sequence that has at least 90%, at least 95%, at least 99%, or 100% identity to any one of
(i) SEQ ID NO:1 and SEQ ID NO:2 respectively;
(ii) SEQ ID NO:3 and SEQ ID NO:4 respectively;
(iii) SEQ ID NO:3 and SEQ ID NO:24 respectively;
(iv) SEQ ID NO:23 and SEQ ID NO:4 respectively;
(v) SEQ ID NO:35 and SEQ ID NO:36 respectively;
(vi) SEQ ID NO:5 and SEQ ID NO:6 respectively;
(vii) SEQ ID NO:5 and SEQ ID NO:27 respectively;
(viii) SEQ ID NO:5 and SEQ ID NO:28 respectively;
(ix) SEQ ID NO:25 and SEQ ID NO:6 respectively;
(x) SEQ ID NO:25 and SEQ ID NO:27 respectively;
(xi) SEQ ID NO:25 and SEQ ID NO:28 respectively;
(xii) SEQ ID NO:26 and SEQ ID NO:6 respectively;
(xiii) SEQ ID NO:26 and SEQ ID NO:27 respectively;
(xiv) SEQ ID NO:26 and SEQ ID NO:28 respectively;
(xv) SEQ ID NO:37 and SEQ ID NO:38 respectively;
(xvi) SEQ ID NO:7 and SEQ ID NO:8 respectively;
(xvii) SEQ ID NO:7 and SEQ ID NO:32 respectively;
(xviii) SEQ ID NO:7 and SEQ ID NO:33 respectively;
(xix) SEQ ID NO:7 and SEQ ID NO:34 respectively;
(xx) SEQ ID NO:29 and SEQ ID NO:8 respectively;
(xxi) SEQ ID NO:29 and SEQ ID NO:32 respectively;
(xxii) SEQ ID NO:29 and SEQ ID NO:33 respectively;
(xxiii) SEQ ID NO:29 and SEQ ID NO:34 respectively;
(xxiv) SEQ ID NO:30 and SEQ ID NO:8 respectively;
(xxv) SEQ ID NO:30 and SEQ ID NO:32 respectively;
(xxvi) SEQ ID NO:30 and SEQ ID NO:33 respectively;
(xxvii) SEQ ID NO:30 and SEQ ID NO:34 respectively;
(xxviii) SEQ ID NO:31 and SEQ ID NO:8 respectively;
(xxix) SEQ ID NO:31 and SEQ ID NO:32 respectively;
(xxx) SEQ ID NO:31 and SEQ ID NO:33 respectively;
(xxxi) SEQ ID NO:31 and SEQ ID NO:34 respectively;
(xxxii) SEQ ID NO:39 and SEQ ID NO:40 respectively;
(xxxiii) SEQ ID NO:9 and SEQ ID NO:10 respectively;
(xxxiv) SEQ ID NO:11 and SEQ ID NO:12 respectively;
(xxxv) SEQ ID NO:13 and SEQ ID NO:14 respectively;
(xxxvi) SEQ ID NO:15 and SEQ ID NO:16 respectively;
(xxxvii) SEQ ID NO:19 and SEQ ID NO:20 respectively;
(xxxviii) SEQ ID NO:21 and SEQ ID NO:22 respectively;
(xxxix) SEQ ID NO:23 and SEQ ID NO:24 respectively;
(xl) SEQ ID NO:41 and SEQ ID NO:42 respectively;
(xli) SEQ ID NO:43 and SEQ ID NO:44 respectively;
(xlii) SEQ ID NO:45 and SEQ ID NO:46 respectively;
(xliii) SEQ ID NO:47 and SEQ ID NO:48 respectively;
(xliv) SEQ ID NO:49 and SEQ ID NO:50 respectively;
(xlv) SEQ ID NO:51 and SEQ ID NO:44 respectively;
(xlvi) SEQ ID NO:53 and SEQ ID NO:52 respectively;
(xlvii) SEQ ID NO:55 and SEQ ID NO:54 respectively;
(xlviii) SEQ ID NO:57 and SEQ ID NO:56 respectively; and
(xlix) SEQ ID NO:59 and SEQ ID NO:58 respectively.

41. The method of any one of claim 1-19 or 37-40, wherein the compA protein is linked to an antigenic protein, optionally via a linker, thereby forming a fusion protein.

42. The method of claim 41, wherein the antigenic protein is selected from HIV Env, RSV F, EBV gp350, CMV gB, CMV UL128, CMV UL130, CMV UL131A, CMV gH, CMV gL, Lyme OspA, Pertussis toxin, Dengue E, SARS S, MERS S, Zaire ebolavirus GP, Sudan ebolavirus GP, Marburg virus GP, Hanta virus Gn, Hanta virus Gc, HepB surface antigen, Measles H, Zika envelope domain III, Malaria CSP, Malaria Pfs25, Nipah virus F, Nipah virus G, Rotavirus VP4, Rotavirus VP8*, hMPV F, hMPV G, PV F, PV HN, MenB fHbp, MenB NadA, coronavirus S protein, coronavirus RBD, and MenB NHBA.

43. The method of claim 41 or 42, wherein the antigenic protein comprises a paramyxovirus and/or penumovirus F protein or an antigenic fragment thereof, optionally a respiratory syncytial virus (RSV) F protein or antigenic fragment thereof, or a human metapneumovirus (hMPV) F protein or an antigenic fragment thereof.

44. The method of claim 41, wherein the antigenic protein comprises a polypeptide comprising an amino acid sequence selected from SEQ ID NOs: 64-136 and 206-209.

45. The method of any one of claims 41-43, wherein the compA protein is linked to the antigenic protein via a peptide linker.

46. The method of claim 45, wherein the peptide linker is a Gly-Ser linker, optionally wherein the Gly-Ser linker is any one of SEQ ID NOs: 213-215.

47. The method of claim 41, wherein the fusion protein comprises an amino acid sequence having at least about 80%, about 85%, about 90%, about 91%, about 92%, about 93%, about 94%, about 95%, about 96%, about 97%, about 98%, about 99%, or about 100% identity to a polypeptide selected from SEQ ID NOs: 137-205.

48. A method of making a nanostructure, comprising adding a fusion protein to a solution comprising a component B (compB) protein under conditions that minimize shear stress, wherein the fusion protein comprises a compA protein linked to an antigenic protein, thereby forming a compA:compB complex.

49. A method of making a nanostructure, comprising adding a fusion protein to a solution comprising a component B (compB) protein under conditions that minimize shear stress, wherein the fusion protein comprises a compA protein linked to an antigenic protein, and wherein the fusion protein comprises an amino acid sequence having at least about 80%, about 85%, about 90%, about 91%, about 92%, about 93%, about 94%, about 95%, about 96%, about 97%, about 98%, about 99%, or about 100% identity to a polypeptide selected from SEQ ID NOs: 137-205, thereby forming a compA:compB complex.

50. The method of any one of claims 1-49, wherein the complex has icosahedral symmetry.

51. A complex produced by the method of any one of claims 1-49.

52. A pharmaceutical composition comprises the complex of claim 51 and a pharmaceutically acceptable diluent.

53. A vaccine comprises the complex of claim 51.

54. A method of treating or preventing a disease or disorder in a subject in need thereof, comprising administering an effective amount of the complex of claim 51, the pharmaceutical composition of claim 52, or the vaccine of claim 53 to the subject.

55. The method of claim 54, wherein the disease or disorder is a viral infection.

56. A method of generating an immune response in a subject in need thereof, comprising administering an effective amount of the complex of claim 51, the pharmaceutical composition of claim 52, or the vaccine of claim 53 to the subject.

57. A kit comprising the complex of claim 51, the pharmaceutical composition or claim 52, or the vaccine of claim 53.

58. Use of the complex of claim 51, the pharmaceutical composition of claim 52, or the vaccine of claim 53 for the method of any one of claims 54-56 or as a medicament.

59. The complex of claim 51, the pharmaceutical composition of claim 52, or the vaccine of claim 53 for use in the method of any one of claims 54-56 or as a medicament.
